(19) United States
(12) Patent Application Publication
(10) Pub. No.: US 2025/0248730 A1
(43) Pub. Date: Aug. 7, 2025

(54) OSCILLATING LATERAL/TRANSVERSE MOTION FOR CUT PATH OF INTRAVASCULAR MEDICAL LEAD REMOVAL DEVICE

(71) Applicant: KONINKLIJKE PHILIPS N.V., EINDHOVEN (NL)

(72) Inventor: CLINT BRUBAKER, COLORADO SPRINGS, CO (US)

(21) Appl. No.: 19/039,897

(22) Filed: Jan. 29, 2025

Related U.S. Application Data
(60) Provisional application No. 63/549,696, filed on Feb. 5, 2024.
(60) Provisional application No. 63/565,197, filed on Mar. 14, 2024.

(51) Int. Cl. A61B 17/32

(52) U.S. Cl. CPC A61B 17/32002

(57) ABSTRACT

An apparatus includes a lead removal device configured to be positioned within a blood vessel of a patient. The device includes a longitudinal axis, a first flexible elongate sheath extending along the longitudinal axis and comprising a cutting tip configured to cut tissue to separate a medical lead implanted in the tissue, and a second flexible elongate sheath extending along the longitudinal axis, wherein the first flexible elongate sheath is positioned inside the first sheath. The cutting tip is configured to have movement relative to the second flexible elongate sheath, including lateral motion in a plane perpendicular to the longitudinal axis.

### CROSS REFERENCE TO RELATED APPLIATIONS

This application claims the benefit of U.S. Provisional Application No. 63/549,696 filed Feb. 5, 2024 and U.S. Provisional Application No 63/565,197 filed Mar. 14, 2024. These applications are incorporated by reference herein.

### TECHNICAL FIELD

The subject matter described herein relates to a cutting tip of a cutting catheter (e.g., a lead removal device). The cutting tip has oscillating lateral/transverse motion to prevent jamming of the cutting tip when tissue that is cut by the cutting tip enters the cutting tip. This apparatus has particular but not exclusive utility for removal of implanted leads from a body lumen of a patient.

### BACKGROUND

Surgically implanted cardiac pacing systems, such as pacemakers and defibrillators, play an important role in the treatment of heart disease. Pacemakers treat slow heart rhythms by increasing the heart rate or by coordinating the heart's contraction for some heart failure patients. Implantable cardioverter-defibrillators stop dangerous rapid heart rhythms by delivering an electric shock. Cardiac pacing systems typically include a timing device and a lead, which are placed inside the body of a patient. One part of the system is the pulse generator containing electric circuits and a battery, usually placed under the skin on the chest wall beneath the collarbone. Another part of the system includes the wires, or leads, which run between the pulse generator and the heart.

These leads must be in contact with heart tissue. To remain attached to the heart muscle, most leads have a fixation mechanism, such as a small screw and/or hooks at the end. Within a relatively short time after a lead is implanted into the body, the body's natural healing process forms scar tissue along the lead and possibly at its tip, thereby fastening it even more securely in the patient's body. Leads usually last longer than device batteries, so leads are simply reconnected to each new pulse generator (battery) at the time of replacement. Although leads are designed to be implanted permanently in the body, occasionally these leads must be removed, or extracted. Leads may be removed from patients for numerous reasons, including but not limited to, infections, lead age, and lead malfunction.

Removal or extraction of the lead may be difficult. As mentioned above, the body's natural healing process forms scar tissue over and along the lead, and possibly at its tip, thereby encasing at least a portion of the lead and fastening it even more securely in the patient's body. In addition, the lead and/or tissue may become attached to the vasculature wall. Both results may, therefore, increase the difficulty of removing the leads from the patient's vasculature.

A variety of tools have been developed to make lead extraction safer and more successful. A mechanical device to extract leads may include one or more flexible tubes called sheaths that passes over the lead and/or the surrounding tissue. One of the sheaths may include a tip having a dilator, a separator and/or a cutting blade, such that upon advancement, the tip cuts or dilates the scar tissue to separate the scar tissue from other scar tissue, including the scar tissue surrounding the lead. In some cases, the tip (and sheath) may also separate the tissue itself from the lead. Once the lead is separated from the surrounding tissue and/or the surrounding tissue is separated from the remaining scar tissue, the lead may be inserted into a hollow lumen of the sheath for removal and/or be removed from the patient's vasculature using some other mechanical devices, such as mechanical traction devices.

Some current lead removal devices include of a handle that transmits torque and rotation to the proximal end of a long shaft. In some versions, the device is used by squeezing the trigger, resulting in an extension and rotation of cutting blades. In other versions, the extension and rotation of the blades is controlled by a motor and electronics in the handle. The cutting blades remove body tissue that would otherwise prevent the extraction of pacemaker leads. The blades of these devices were designed to cut through the lesion material and allow the lead to be extracted. The design of the blades has a ring of cutting teeth around the outer diameter that then tapers down to the inner diameter of the cutter.

However, one of the main issues encountered in using such lead removal devices is stuck or jammed leads. A stuck or jammed lead occurs during a procedure when the device cuts through a lesion adhered to a lead. After cutting partway or through the lesion, the lesion and lead get stuck within the device, leading to the inability to pull out the lead from the device and/or preventing further cutting of the lesion.

The information included in this Background section of the specification, including any references cited herein and any description or discussion thereof, is included for technical reference purposes only and is not to be regarded as subject matter by which the scope of the disclosure is to be bound.

### SUMMARY

The present disclosure provides an intraluminal cutting device with several advantageous features. One such feature is an offset blade cutting tip. Oscillating the cutter around a point allows the effective cutting area to increase proportionately to the magnitude of the offset for the oscillation. Oscillating the cutter allows the blade to act as a larger cutter while keeping the material plug smaller than the inner diameter of the blade. This can be accomplished with a cutting tip that purposely oscillates around a center point, not at the concentric, geometric center of the blade's cutter in a mechanical lead removal device. This reduces or eliminated the compression the tissue experiences as the cutting tip is advanced over the implanted lead, and thus reduces or eliminated compression-related friction on the intraluminal cutting device, limiting the likelihood that the cutting device will jam.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes an apparatus that includes a lead removal device configured to be positioned within a blood vessel of a patient. The lead removal device may include: a longitudinal axis; a first flexible elongate sheath extending along the longitudinal axis and including a cutting tip configured to cut tissue to separate a medical lead implanted in the tissue; and a second flexible elongate sheath extending along the longitudinal axis, where the first flexible elongate sheath is positioned inside the second flexible elongate sheath, where the cutting tip is configured to have movement relative to the second flexible elongate sheath, where the movement may include lateral motion in a plane perpendicular to the longitudinal axis. Other examples of this aspect include corresponding computer systems, apparatus, and computer programs recorded on one or more computer storage devices, each configured to perform the actions of the methods.

Implementations may include one or more of the following features. In some aspects, the movement may include rotational motion around the longitudinal axis and longitudinal motion along the longitudinal axis. In some aspects, the lateral motion of the cutting tip is configured to cause the rotational motion of the cutting tip to be non-concentric relative to the second flexible elongate sheath. In some aspects, the lead removal device further may include a cam pin may include a first end fixedly coupled to the cutting tip. In some aspects, the second flexible elongate sheath may include a band, where the band may include a cam path such that the cam path is positioned radially outward of the first end of the cam pin, where an opposite, second end of the cam pin is configured to travel along the cam path. In some aspects, at least one of the cam path or the second end of the cam pin is sized and shaped to prevent separation between the cam pin and the cam path. In some aspects, the cutting tip may include a lumen configured to receive at least of one of the tissue after being cut or the medical lead after being separated such that at least one of the tissue or the medical lead that enters the lumen has the lateral motion. In some aspects, the lateral motion of the cutting tip is configured to cause a diameter of the tissue entering the lumen to be smaller than an inner diameter of the cutting tip. Implementations of the described techniques may include hardware, a method or process, or computer software on a computer-accessible medium.

One general aspect includes a lead removal device. The lead removal device includes a flexible elongate member configured to be positioned within a blood vessel of a patient, where the flexible elongate member includes a proximal portion and a distal portion. The device also includes an outer band fixedly positioned at the distal portion of the flexible elongate member and may include an inner surface having an inner diameter and a cam slot. The device also includes a cutting tip positioned at least partially within the outer band and configured to rotate with respect to the outer band, where the cutting tip may include: a blade configured to cut tissue associated with the blood vessel for removal of an electrical lead implanted in the tissue from a body of a patient; an outer wall surface defining an outer diameter that is smaller than the inner diameter of the outer band, such that a gap exists between the outer wall surface and the inner surface of the outer band; an inner wall surface defining a lumen with an inner diameter, where the lumen is configured to receive the electrical lead and the tissue cut by the blade; and a guide pin fixedly attached to the outer wall surface and configured to slidably engage with the cam slot such that, proximate to the guide pin, the outer wall surface is in contact with the inner surface of the outer band, where rotation of the cutting tip within the outer band causes the guide pin to slide within the cam slot, such that a center of rotation of the cutting tip is not aligned with a geometric center of the cutting tip, causing the cutting tip to move laterally as it rotates, where a cutting edge of the blade is aligned with the inner wall surface, such that a diameter of the tissue cut by the blade and entering the lumen is smaller than the inner diameter. Other examples of this aspect include corresponding computer systems, apparatus, and computer programs recorded on one or more computer storage devices, each configured to perform the actions of the methods.

Implementations may include one or more of the following features. In some aspects, a diameter of a region of tissue shredded by the cutting tip is equal to the inner diameter of the outer band. In some aspects, the cutting tip is configured such that at least a portion of tissue shredded by the cutting tip is received into the lumen. In some aspects, the blade may include a plurality of teeth and a plurality of serrations, where a distal end of each tooth may include a cutting edge. In some aspects, each tooth of the plurality of teeth may include a chamfered trapezoidal surface, where each serration of the plurality of serrations may include a chamfered curved surface. In some aspects, the lead removal device may include: a handle coupled to the proximal portion of the flexible elongate member; and, a trigger coupled to the handle and configured such that actuation of the trigger causes the cutting tip to rotate. In some aspects, the trigger is coupled to a motor and a battery such that actuation of the trigger causes the motor to rotate the cutting tip. In some aspects, the trigger is coupled to the motor via a processor. In some aspects, the lead removal device may include an outer jacket slidably coupled to the flexible elongate member such that when the outer jacket is advanced to a distal position, the blade is covered by the outer jacket, and when the outer jacket is retracted to a proximal location, the blade is not covered by the outer jacket. In some aspects, the cam slot is curved such that when the rotation of the cutting tip within the outer band causes the guide pin to slide within the cam slot, causing the cutting tip to move longitudinally. Implementations of the described techniques may include hardware, a method or process, or computer software on a computer-accessible medium.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the intraluminal cutting device, as defined in the claims, is provided in the following written description of various aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a diagrammatic schematic view of an intraluminal cutting device or surgical device, such as a cutting catheter device, according to aspects of the present disclosure.

FIG. 2 is a side perspective view of an example surgical device, according to aspects of the present disclosure.

FIG. 3 is a side cross-sectional view of a distal portion of an example sheath assembly, according to aspects of the present disclosure.

FIG. 4 is a perspective side view of the distal end of an example sheath assembly, according to aspects of the present disclosure.

FIG. 5 is a perspective side view of the distal end of an example sheath assembly, according to aspects of the present disclosure.

FIG. 6 is a diagrammatic view of a surgical device that has been introduced into a body lumen of a patient to remove an implanted lead, according to aspects of the present disclosure.

FIG. 7 is a side cross-sectional view of an example sheath assembly removing a lead from a body lumen, according to aspects of the present disclosure.

FIG. 8 is a side front perspective view of an example cutting tip, according to aspects of the present disclosure.

FIG. 9 is a schematic cross-sectional view of the reciprocating motion of a cutting tip of the distal portion of an example sheath assembly, according to aspects of the present disclosure.

FIG. 10 is a schematic cross-sectional view of the reciprocating motion of a cutting tip of the distal portion of the example sheath assembly of FIG. 9, according to aspects of the present disclosure.

FIG. 11 is a schematic cross-sectional view of the reciprocating motion of a cutting tip of the distal portion of the example sheath assembly of FIG. 10, according to aspects of the present disclosure.

FIG. 12 is a side cross-sectional view of the cutting tip of FIG. 8, according to aspects of the present disclosure.

FIG. 13 is a schematic, diagrammatic, side cross-sectional view of an example cutting tip cutting into tissue surrounding a lead, according to aspects of the present disclosure.

FIG. 14 is a graph showing a curve of compressive friction force as a function of the cut depth of the cutting tip into the tissue surrounding the lead, according to aspects of the present disclosure.

FIG. 15 is an end view of an example cutting tip rotating along an oscillating cut path, according to aspects of the present disclosure.

FIG. 16 is an end view of an example cutting tip rotating along an oscillating cut path, according to aspects of the present disclosure.

FIG. 17 is a schematic, diagrammatic, side cross-sectional view of an example oscillating cutting tip cutting into tissue surrounding a lead, according to aspects of the present disclosure.

FIG. 18 is a schematic, diagrammatic end view of an example cut region created by an oscillating cut path, according to aspects of the present disclosure.

FIG. 19 is a graph showing a curve of compressive friction force as a function of the cut depth of the oscillating cutting tip into the tissue surrounding the lead, according to aspects of the present disclosure.

FIG. 20 is a diagrammatic schematic view of an intraluminal cutting device or surgical device, such as a cutting catheter device, according to aspects of the present disclosure.

FIG. 21 is a top side perspective view of at least a portion of an example cutting tip assembly, according to aspects of the present disclosure.

FIG. 22 is a top side perspective view of an example cutting tip, according to aspects of the present disclosure.

FIG. 23 is a lateral cross-sectional view of an example cutting tip, according to aspects of the present disclosure.

FIG. 24 is an enlarged view of the guide pin of FIG. 23, according to aspects of the present disclosure.

FIG. 25 is an end view of an example cutting tip, according to aspects of the present disclosure.

FIG. 26 is a top side perspective view of an example outer band, according to aspects of the present disclosure.

FIG. 27 is a top side perspective cross-sectional view of an example outer band, according to aspects of the present disclosure.

FIG. 28 is a lateral cross-sectional view of an example outer band, according to aspects of the present disclosure.

FIG. 29 is an enlarged view of the cam slot of FIG. 28, according to aspects of the present disclosure.

FIG. 30 is a lateral cross-sectional view of a cutting tip positioned within an example outer band, according to aspects of the present disclosure.

FIG. 31 is an enlarged view of the guide pin and cam slot of FIG. 30, according to aspects of the present disclosure.

FIG. 32 is an end view of an example cutting tip positioned within an example outer band, according to aspects of the present disclosure.

FIG. 33 is an end cross-sectional view of an example cutting tip positioned within an example outer band, according to aspects of the present disclosure.

FIG. 34 is a cross-sectional view of an example guide pin, according to aspects of the present disclosure.

FIG. 35 is a cross-sectional view of an example guide pin, according to aspects of the present disclosure.

FIG. 36 is a cross-sectional view of an example spool-shaped guide pin, according to aspects of the present disclosure.

FIG. 37 is a cross-sectional view of an example guide pin, according to aspects of the present disclosure.

FIG. 38 is an end cross-sectional view of an example cutting tip inside an example outer band, according to aspects of the present disclosure.

FIG. 39 is a schematic diagram of a processor circuit, according to aspects of the present disclosure.

### DETAILED DESCRIPTION

Currently, mechanical products are used in lead management/removal procedures. These devices may include a handle that transmits torque and rotation to the proximal end of a cutting catheter, whose distal end includes a cylindrical cutting tip with a sharp or serrated lip forming one or more blades. In some aspects, the device is actuated by applying a squeezing force to the trigger, resulting in an extension and rotation of the cutting blades. In other aspects, the extension and rotation of the blades is controlled by a motor and electronics in the handle of the device. The cutting blades remove body tissue that would otherwise prevent the extraction of pacemaker leads. The blades of these devices are designed to cut through the lesion material and allow the lead to be extracted. The design of the blades can include a ring of cutting teeth around the outer diameter of the cutting tip that then tapers down (e.g., at a 60-degree angle) to the inner diameter of the cutting tip.

One issue encountered in using such cutting tips is sticking or jamming of electrical leads within the cutting tip. A stuck or jammed lead occurs during a procedure when the device cuts through a lesion adhered to a lead. After the tip has cut partway through the lesion, the lesion and lead get stuck within the tip, leading to an inability to pull out the lead from the device and/or preventing further cutting of the lesion.

One cause of this problem is with the current blade profile. Because of the inward chamfer of the tip, the outer diameter of the plug of material that is cut and pulled into the lumen of the cutter/shaft is larger than the inner diameter of the cylindrical cutting tip. In one non-limiting example, on some devices that have encountered jamming issues, the cut plug reduces from the cut diameter of 0.205 inches to 0.171 inches in diameter of the inner cutting tip lumen, which equates to a compression of 16.6%. In some devices, the compression is constant through the shaft due to the straight shaft design, where the inner diameter is consistent throughout the entire device. This leads to an effect where the further the device cuts into the lesion material, the more frictional resistance it encounters. However, in other exemplary devices, the plug only has to reduce from the cut diameter of 0.191 to 0.171 inches, thus compressing 10.5%.

In addition, in some devices, proximal of the cutting tip, the catheter shaft opens up to a diameter of 0.183 inches, bringing the plug compression down to 4.2%. It has been observed that such devices can cut much farther in clinical use and testing than devices with a greater degree of compression. Accordingly, a need exists to reduce or eliminate this compressive action during cutting on mechanical lead removal devices, to improve the performance of devices and to reduce clinical complaints. The present disclosure provides an intraluminal cutting device with several advantageous features.

One such feature is an offset blade cutting tip. Oscillating the cutter around a point allows the effective cutting area to increase proportionately to the magnitude of the offset for the oscillation. Oscillating the cutter allows the blade to act as a larger cutter while keeping the material plug smaller than the inner diameter of the blade. This can be accomplished with a cutting tip that purposely oscillates around a center point, not at the concentric, geometric center of the blade's cutter in a mechanical lead removal device. This reduces or eliminates the compression the tissue experiences as the cutting tip is advanced over the implanted lead, and thus reduces or eliminated compression-related friction on the intraluminal cutting device, limiting the likelihood that the cutting device will jam.

To build the offset blade cutter, existing band designs can be modified for machining to have alternating offset blades. The new cutter design can then be incorporated into novel or existing lead removal devices.

The present disclosure substantially aids a clinician in removing an implanted lead from a body lumen of a patient, by providing a cutting tip that reduces or eliminates friction caused by compression of the tissue plug as it advances through the cutting tip. The system may include cutting tips with specialized designs. Implemented on a cutting catheter in mechanical communication with a handle assembly, the intraluminal cutting device disclosed herein may provide both time savings and an improvement in the safety and precision of lead removal procedures. This improved lead removal workflow transforms a slow, painstaking process into one where the risk of friction-related jamming is greatly reduced, without the normally routine need to halt the procedure and remove the cutting catheter. This unconventional approach improves the functioning of the lead removal system, by allowing a single cutting catheter to cut much farther into scar tissue without jamming.

The devices, systems, and methods described herein can include one or more features described in U.S. Pat. No. 10,314,615 and titled “Medical device for removing an implanted object”, U.S. Pat. No. 9,980,743 and titled “Medical device for removing an implanted object using laser cut hypotubes”, U.S. Pat. No. 10,136,913 and titled “Multiple configuration surgical cutting device”, and U.S. Pat. No. 10,993,741 and titled “Surgical cutting device with shield drive mechanism”, each of which is hereby incorporated by reference in their entirety as though fully set forth herein.

Portions of the system disclosed herein may be implemented as a set of logical branches and mathematical operations, whose outputs are viewable on a display, and operated by a control process executing on a processor that accepts user inputs from a trigger, touchscreen interface, or other user interface, and that is in communication with one or more motors controlling rotation of the cutting tip. In that regard, the control process performs certain specific operations in response to different inputs or selections made by a user at different times. Certain structures, functions, and operations of the processor, display, sensors, and user input systems are known in the art, while others are recited herein to enable novel features or aspects of the present disclosure with particularity.

These descriptions are provided for exemplary purposes only, and should not be considered to limit the scope of the disclosure. Certain features may be added, removed, or modified without departing from the spirit of the claimed subject matter.

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one aspect may be combined with the features, components, and/or steps described with respect to other aspects of the present disclosure. For the sake of brevity, however, the numerous iterations of these combinations will not be described separately.

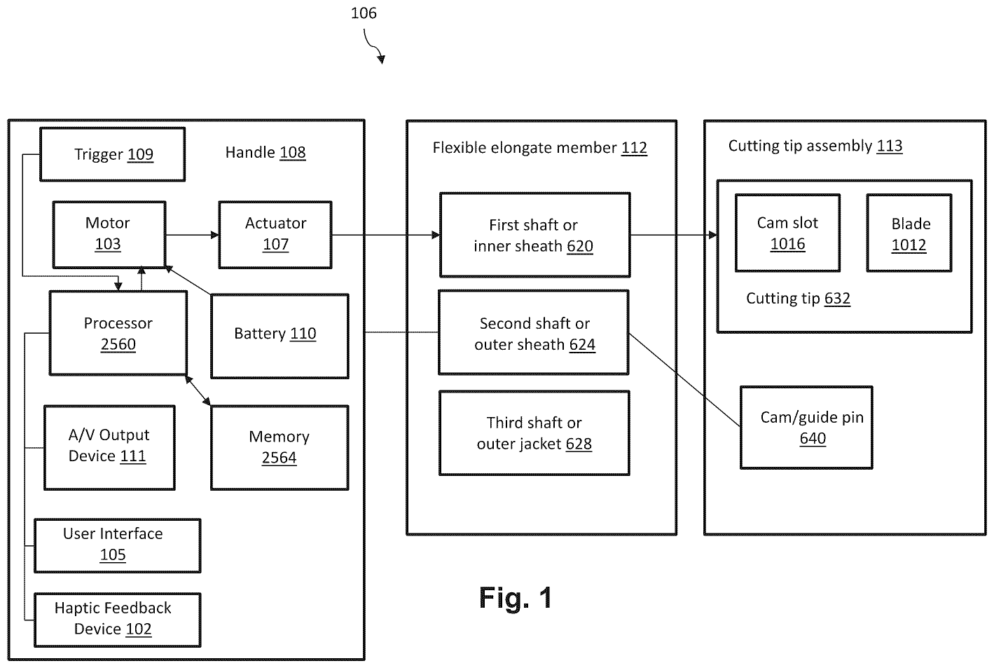

FIG. 1 is a diagrammatic schematic view of an intraluminal cutting device 106 or surgical device 106, such as a cutting catheter device, according to aspects of the present disclosure. The intraluminal cutting device or surgical device 106 includes a handle 108, a flexible elongate member or sheath assembly 112, and a cutting tip assembly 113.

The handle 108 may include a trigger 109, one or more motors 103, an actuator 107, a processor 2560, a battery 110, an audiovisual (A/V) output device 111 (e.g., a display screen, a set of indicator lights, etc.), and a user interface 105 (e.g., a touchscreen, one or more buttons, switches, dials, etc.).

The flexible elongate member or sheath assembly 112 may include a rotatable first shaft or flexible inner sheath 620 coupled to the actuator 107, a fixed second shaft or flexible outer sheath 624 that surrounds the flexible inner sheath 620, and a translatable third shaft or outer jacket 628 that surrounds the flexible outer sheath 624.

In an example, the actuator 107 be or include a power train assembly with one or more gears coupling the 103 motor to the first (inner) shaft or flexible inner sheath 620. A press of the trigger 109 may send a signal to the processor 2560, which then activates the motor 103, which is powered by the battery 110 and transmits rotational motion through the actuator 107 to the flexible inner sheath 620, which then rotates along with the actuator. The flexible outer sheath 624 may be fixed to the handle 108, such that it remains fixed while the flexible inner sheath 620 rotates within it.

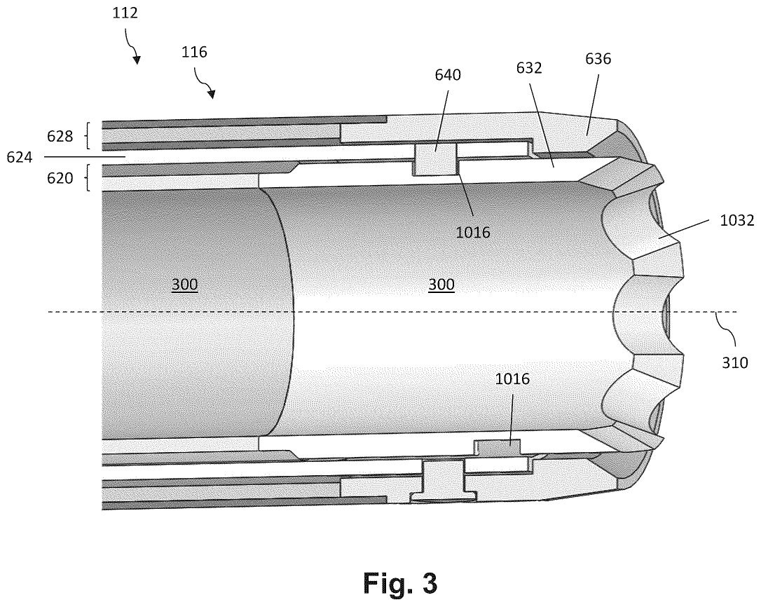

The cutting tip assembly 113 may for example include a cutting tip 632 with a cam slot 1016 and blade 1012. The cutting tip 632 is fixedly attached to the flexible inner shaft 620, such that rotation of the flexible inner shaft 620 causes rotation of the cutting tip 632. A cam pin, guide pin, or cam guide pin 640 is fixedly attached to the flexible outer sheath 624, but fits within the cam slot 1016, such that rotation of the cutting tip 632 can also drive axial or longitudinal motion of the cutting tip (e.g., along a longitudinal axis of the lead removal device, flexible elongate member, and/or cutting tip, such as longitudinal axis 310 in FIG. 3), as described below.

The third shaft or outer jacket 628 surrounds the second shaft or outer sheath 624, and is translatably movable along it, such that a distal portion of the outer jacket 628 can be advanced to cover the blade 1012 of the cutting tip 632, as described below.

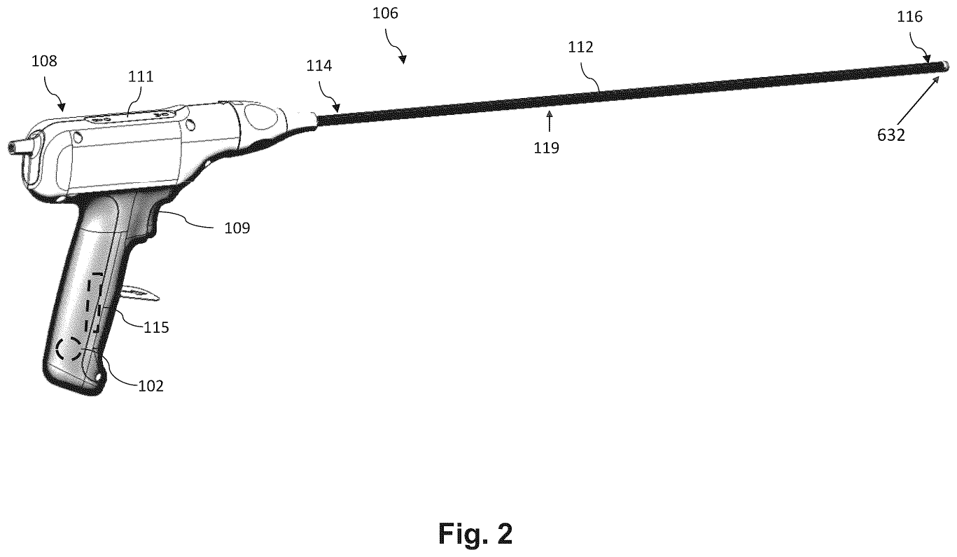

FIG. 2 is a side perspective view of an example surgical device 106, according to aspects of the present disclosure. The surgical device 106 includes a sheath assembly 112 that can be inserted into a body lumen 334 of a patient 104 (see FIG. 6). The sheath assembly includes a proximal portion 114 and a distal portion 118, separated by a working length 119 that is sufficient to perform the tasks described herein. The distal portion 118 includes a movable cutting tip 632.

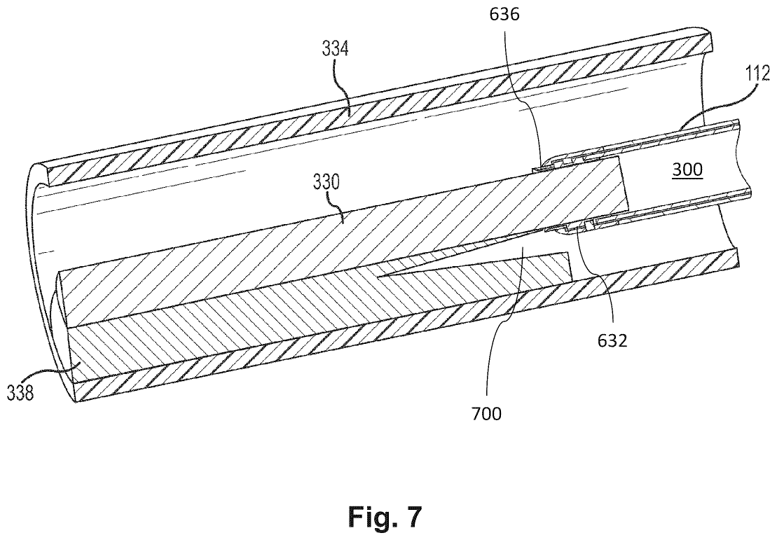

The sheath assembly 112 can surround an implanted lead 330 (see FIG. 7), such as a lead running along the left innominate vein past the superior vena cava (SVC) and connected into, or about, the right ventricle of the heart. Upon surrounding the lead 330 with the sheath assembly 112, the user of the surgical device 106 may actuate the handle assembly 108 (e.g., with a trigger 109), thereby actuating the cutting tip 632 located at the distal end of the sheath assembly 112, as described below. The actuated cutting tip 632, can then separate and/or cut the tissue surrounding an implanted lead within the body lumen of the patient.

Depending on the implementation, the handle assembly 108 may also include audiovisual (A/V) feedback indicators 111, a controller printed circuit board assembly (PCBA) 115, and a haptic feedback device 102.

FIG. 3 is a side cross-sectional view of a distal portion 116 of an example sheath assembly 112, according to aspects of the present disclosure. The distal portion 116 of the sheath assembly 112 includes an outer band 636 fixedly attached to a flexible outer jacket 628, and a cutting tip 632 fixedly attached to a flexible inner sheath 620. In the example shown in FIG. 3, the flexible outer sheath 624 surrounds the flexible inner sheath 620, and the outer band 636 surrounds the cutting tip 632. The cutting tip 632 and flexible inner sheath 620 together define an inner lumen 300. A guide pin 640 is fixedly attached to the outer band 636. The cutting tip 632 is rotatably attached to the outer band 636 via the guide pin 640 that rests in a channel or can slot 1016. Activation of the trigger 109 of the handle assembly 108 (see FIG. 2) causes the flexible inner sheath 620 to rotate, whereas the flexible outer sheath 624 is rotationally fixed. The channel or cam slot 1016 is formed in the cutting tip 632 in a profile that varies in longitudinal distance with different radial positions, such that when the flexible inner sheath 620 is rotated while the flexible outer sheath 624 is rotationally fixed, the guide pin 640 travels through the cam slot 1016, causing the flexible inner shaft 620 and the cutting tip 632 to translate longitudinally as they rotate, as will be shown in greater detail below.

Depending on the profile of the cam slot 1016, a serrated blade or cutting surface 1012 of the cutting tip 632 may thus extend from and retract into the outer band 636 multiple times upon actuation of the trigger of the handle assembly. Depending on the implementation, the blade 1012 may rotate in a clockwise direction, a counterclockwise direction, or may oscillate between the two. When the clinician releases the trigger of the handle assembly, the blade 1012 of the cutting tip 632 may retract within the outer band 636, thereby allowing the clinician to force and advance the distal portion of the sheath assembly against additional uncut tissue, without engagement of the tissue by the blade 1012 of the cutting tip 632. The clinician may repeat the actuation step, thereby causing the blade 1012 of the cutting tip 632 to extend distally beyond the outer band 636 to cut the adjacent tissue. Each time actuation occurs, the proximal portion of the implanted lead and/or surrounding tissue enters further into the central lumen 300 of the sheath assembly 112. This process can be repeated until the surrounding tissue is completely or substantially dilated, and the implanted lead is separated and/or cut from the tissue. At that time, the implanted lead may safely be removed from the patient.

Also visible is a longitudinal axis 310 of the lead removal device, flexible elongate member, and/or the cutting tip.

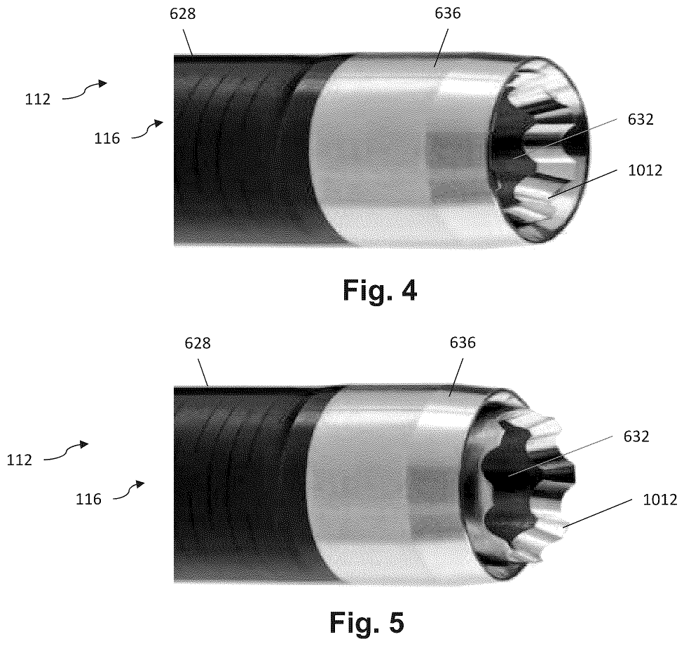

FIG. 4 is a perspective side view of the distal end of an example sheath assembly 112, according to aspects of the present disclosure. In this example, the blade 1012 of the cutting tip 632 is retracted inside the outer band 636. Also visible is the flexible outer jacket 628. In this configuration, the outer band 636 can be advanced against tissue in order to dilate or separate it without cutting (e.g., to separate the tissue from an implanted lead).

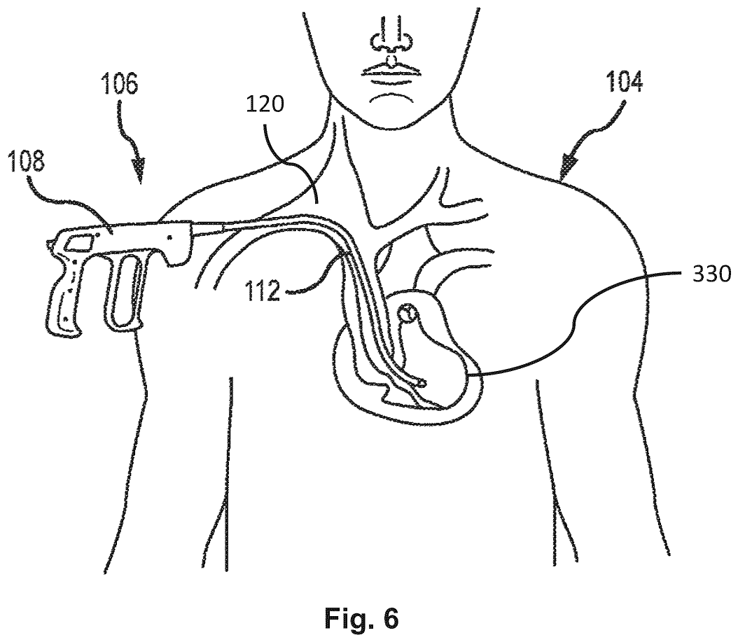

FIG. 5 is a perspective side view of the distal end of an example sheath assembly 112, according to aspects of the present disclosure. In this example, the blade 1012 of the cutting tip 632 is extended beyond the distal end of the outer band 636. Also visible is the flexible outer jacket 628. In this configuration, the blade 1012 can be advanced against tissue in order to cut it (e.g., to separate the tissue from an implanted lead).

FIG. 6 is a diagrammatic view of a surgical device 106 that has been introduced into a body lumen 334 of a patient 104 to remove an implanted lead 330, according to aspects of the present disclosure. The lead 330 may be surrounded by or embedded in tissue, which may be separated from the lead by the surgical device 106 as described herein. The lead may then be drawn into a lumen of the surgical device (e.g., lumen 300 of FIG. 3) for removal from the body lumen 334.

FIG. 7 is a side cross-sectional view of an example sheath assembly 112 removing a lead 330 from a body lumen 334, according to aspects of the present disclosure. After being implanted in the body lumen 334 for a period of time, the lead 330 may be partially or completely surrounded by tissue 338 (e.g., scar tissue) that has grown over the lead 330 within the body lumen 334. The tissue 338 may be attached or adhered to both the lead 330 and the wall of the body lumen 334, thus making the lead 330 difficult to safely remove from the body lumen 334.

In order to remove the lead safely, a clinician may advance the sheath assembly 112 over a portion the lead 330 such that the lead 330 at least partially enters the lumen 300 of the sheath assembly 112. The sheath assembly may then be further advanced until it contacts the tissue 338, at which point the outer band 636 may be used to dilate the tissue, and/or the cutting tip 632 may be extended distal of the outer band such that the blade of the cutting tip (e.g., blade 1012 of FIG. 5) rotates and/or translates in contact with the tissue 338, thus cutting the tissue. Through a combination of dilation and cutting, the sheath assembly 112 may thus form a gap 700 between the tissue 338 and the lead 330. In cases where the tissue 338 completely surrounds the lead 330, the gap 700 may for example be a circular or cylindrical gap that is roughly concentric with the sheath assembly 112. When the gap 700 had been advanced past either an end of the lead 330 or an end of the tissue overgrowth 338, the lead may no longer be adhered, and may be safely removable from the body lumen 334.

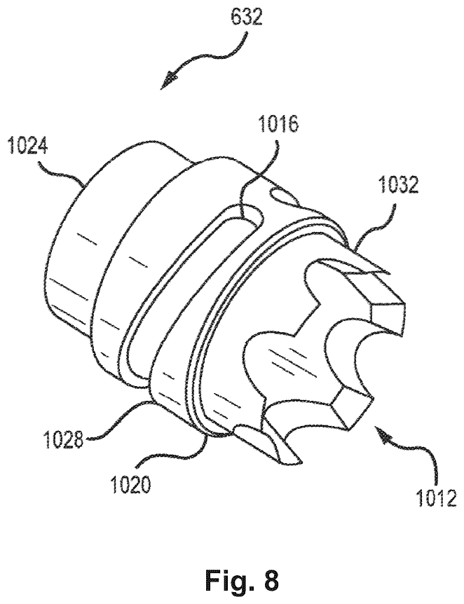

FIG. 8 is a side front perspective view of an example cutting tip 632, according to aspects of the present disclosure. The cutting tip 632 has a generally hollow cylindrical shape. The cutting tip 632 comprises a proximal portion 1024, an intermediate portion 1028, and a distal portion 1032. The outside diameter of the proximal portion 1024 is sized to allow it to be inserted to and/or engage (or otherwise attached to) the interior diameter of the flexible inner sheath (e.g., flexible inner sheath 620 of FIG. 3). The distal end of cutting tip 632 comprises a blade or cutting surface 1012, which may for example have a serrated, sharp blade profile. The intermediate portion 1028 comprises a channel or cam slot 1016 cut within its exterior surface.

As the inner flexible sheath rotates and translates within the outer sheath (e.g., flexible outer sheath 624 of FIG. 3), the outer sheath and pin may remain stationary. If so, the inner sheath, which is connected to cutting tip 632, forces the cutting tip 632 to rotate. The cam slot 1016 engages the guide pin, and the shape and profile of the cam slot 1016 controls the rate and distance with which the cutting tip 632 travels longitudinally. That is, the configuration of the cam slot 1016 controls the cutting tip's direction and amount of longitudinal travel as the cutting tip 632 is rotated, such as moving distally toward an extended position and/or proximally toward a retracted position, while the cutting tip 632 rotates in either a clockwise or counter-clockwise direction.

In some aspects, the cutting tip 632 may also comprise a step up 1020 such that the outer diameter of the intermediate portion 1028 is greater than the outer diameter of the distal portion 1032, thus preventing the intermediate portion 1028 from fitting within the inner diameter of the inner sheath. As the cutting tip 632 rotates, and the blade or cutting surface 1012 extends beyond the distal end of the outer band into an extended position, the step up 1020 of the cutting tip 632 contacts the abutment of the outer band, thereby limiting the distance that the cutting tip 632 may travel and/or preventing the cutting tip 632 from exiting or extending beyond the distal tip of the outer sheath assembly, particularly the outer band, in the event that the guide pin is sheared.

The profile of the cam slot in the cutting tip may have various configurations, such as those disclosed in U.S. patent application Ser. No. 13/834,405 filed Mar. 15, 2013 and entitled Retractable Blade For Lead Removal Device, which is hereby incorporated herein by reference in its entirety as though fully set forth herein. For example, the cam slot 1016 may have a substantially linear profile, a substantially sinusoidal profile, or a combination of linear and non-linear profiles. Additionally, the cam slot 1016 may have an open and continuous configuration, thereby allowing the cutting tip to continuously rotate, or the cam slot may have a closed and discontinuous configuration such that when the cutting tip reaches its fully extended position, the trigger of the handle assembly may be released or reversed so that the cutting tip returns to initially retracted position before being re-actuated. For instance, the cam slot 1016 in FIG. 8 is discontinuous because the cam slot does not travel around the entire circumference of the exterior of the cutting tip 632.

Although certain figures in this disclosure only illustrate either the open or closed cam slot configuration, either configuration may be used with any of the aspects disclosed and/or discussed herein and are considered within the scope of this disclosure. Furthermore, various types of cam slots 1016, such as a partial lobe cam (which includes a cam slot 1016 surrounding less than 360 degrees of the circumference of the exterior surface of the cutting tip 632), a single lobe cam (which includes a cam slot 1016 surrounding 360 degrees of the circumference of the exterior surface of the cutting tip 632), a double lobe cam (which includes a cam slot 1016 surrounding 720 degrees of the circumference of the exterior surface of the cutting tip 632) and/or other multiple lobe cams.

The distal end of cutting tip 632 may comprise a cutting surface 1012 having different blade profiles, such as those disclosed in U.S. patent application Ser. No. 13/834,405 filed Mar. 15, 2013 and entitled “Retractable Blade For Lead Removal Device”, which is hereby incorporated herein by reference in its entirety as though fully set forth herein. For example, the plane of the blade or cutting surface 1012 of the distal end 1032 of the cutting tip 632 depicted in the figures of this disclosure is parallel to the plane of the proximal end 1024 of the cutting tip 632. The plane of the cutting surface, however, may be offset (0 degrees to 90 degrees) from the plane of the proximal end 1024 of the cutting tip 623. Also, as discussed above, the profile of the cutting surface 1012 shown in FIG. 8 includes a plurality of serrations. However, depending on the implementation, the profile of the cutting surface 1012 need not be serrated, and may comprise other configurations, such as a constant and/or smooth sharp profile. The profile of the cutting surface 1012 in FIG. 8 includes 6 serrations. However, it may be desirable to have other numbers of serrations, such as 4, 5, 7, 8, 10, or more serrations. Furthermore, the serrations may comprise a myriad of different shapes and configurations, including but not limited to any variation of a square, rectangle, rhombus, parallelogram, trapezoid, triangle, circle, ellipse, kite, etc.

As discussed above, FIG. 8 depicts the intermediate portion 1028 of the cutting tip 632 having a cam slot (or channel) 1016 cut within its exterior surface, as a means of controlling longitudinal motion of the cutting tip 632 as the cutting tip 632 is rotated. It should be understood that this mechanism is presented here for exemplary purposes, and that other means of rotating and/or translating the cutting tip 632 may be used instead or in addition, without departing from the spirit of the present disclosure, so long as at least some of the methods described herein can be performed.

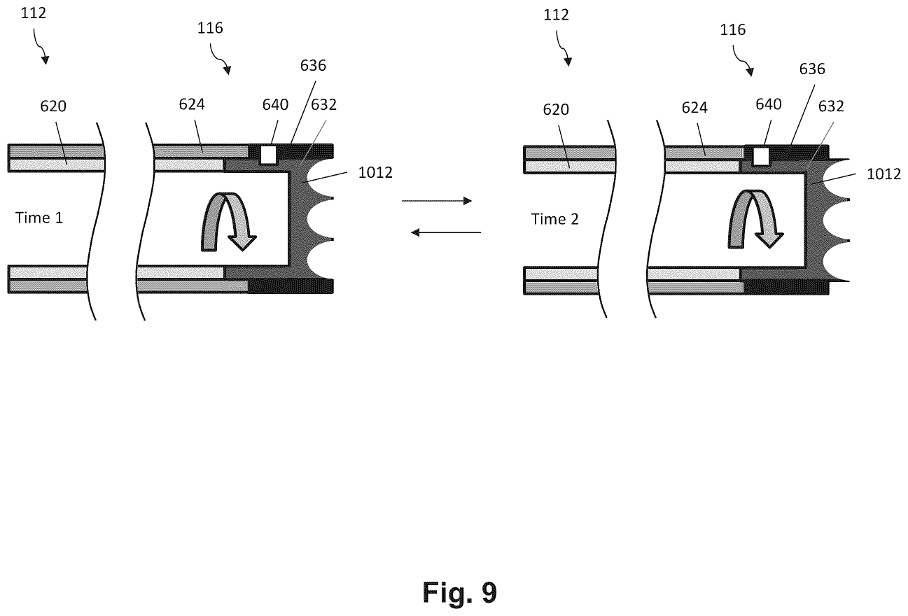

FIG. 9 is a schematic cross-sectional view of the reciprocating motion of a cutting tip 632 of the distal portion 116 of an example sheath assembly 112, according to aspects of the present disclosure. Visible is the guide pin 640, which is fixedly attached to the outer band 636, which is fixedly attached to a distal end of the flexible outer sheath 624. The flexible outer sheath 624 surrounds the flexible inner sheath 620, to whose distal end the cutting tip 632 is fixedly attached. Due to the motion of the guide pin 640 in the cam slot 1016 as the flexible inner sheath and cutting tip 632 (as described above), the cutting tip 632 may move longitudinally as it rotates, such that at a first time (“Time 1”) the blade 1012 of the cutting tip 632 is in a retracted position within the outer band 636, while at a second time the blade 1012 of the cutting tip 632 is in an extended or cutting position wherein the blade 1012 projects beyond a distal end of the outer band 636. Depending on the implementation, rotation of the cutting tip 632 relative to the outer band 636 may cause the blade 1012 to oscillate between the extended and retracted positions, either by continuous rotation in one direction (e.g., clockwise or counterclockwise) or by oscillating rotation in alternating directions. In some examples, the retracted position may represent a “home” position for the blade 1012, such that when the trigger of the handle assembly is released, the flexible inner sheath 620 and the cutting tip 632 are automatically rotated to a “home” clock angle wherein the cutting tip 632 is translated to a longitudinal position wherein the blade 1012 of the cutting tip 632 is behind the outer band 636 and thus protected from cutting tissues of the patient.

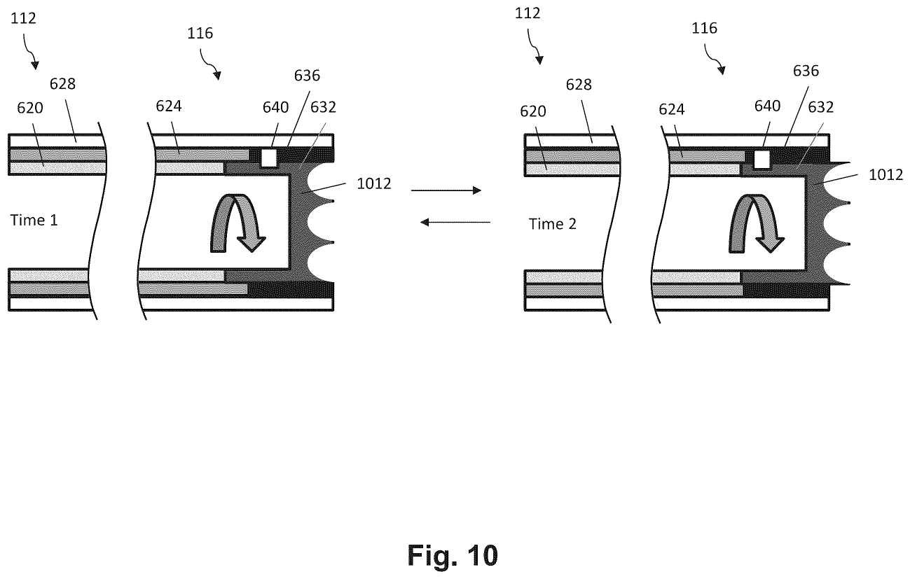

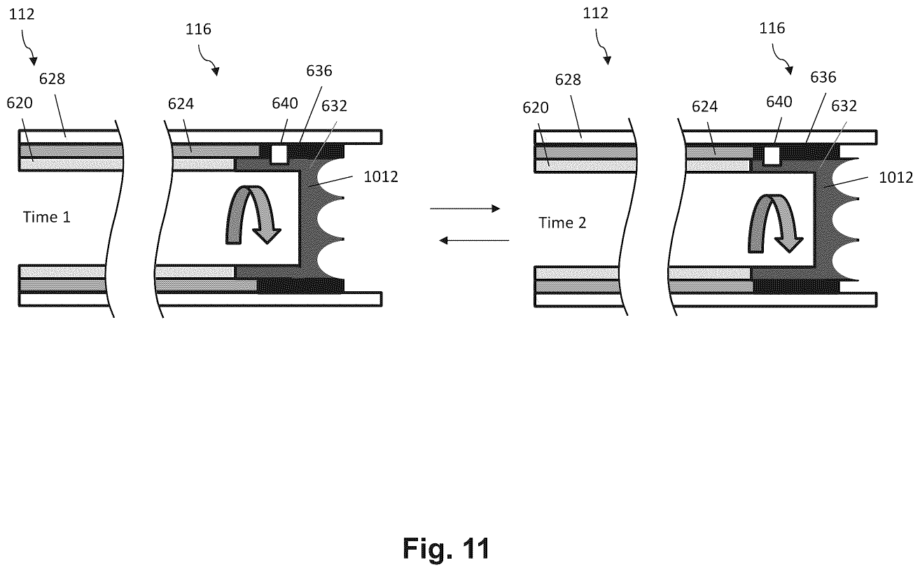

FIG. 10 is a schematic cross-sectional view of the reciprocating motion of a cutting tip 632 of the distal portion 116 of the example sheath assembly 112 of FIG. 9, according to aspects of the present disclosure. Visible are the guide pin 640, outer band 636, flexible outer sheath 624, flexible inner sheath 620, cutting tip 632, and blade 1012. Also visible in FIG. 10 is an outer jacket or guard 628, which surrounds the flexible outer sheath 624. The outer jacket or guard 628 may extend from a proximal end to a distal end of the sheath assembly 112, and may be manually extendable or retractable by the clinician, such that in its fully retracted position (shown here in FIG. 10) the outer jacket or guard 628 does not extend beyond the distal end of the outer band 636. In a fully extended position (shown below in FIG. 11), a distal end of the outer jacket or guard 628 may extend distal of the distal end of the outer band 636. In this configuration, the reciprocating action of the cutting tip 623 and blade 1012 can proceed as described above in FIG. 9.

FIG. 11 is a schematic cross-sectional view of the reciprocating motion of a cutting tip 632 of the distal portion 116 of the example sheath assembly 112 of FIG. 10, according to aspects of the present disclosure. Visible are the guide pin 640, outer band 636, flexible outer sheath 624, flexible inner sheath 620, cutting tip 632, and blade 1012. Also visible in FIG. 11 is the outer jacket or guard 628, which surrounds the flexible outer sheath 624. In the example of FIG. 11, the outer jacket or guard 628 is in an extended position, such that the distal end of the outer jacket or guard 628 extends distal of the distal end of the outer band 636, by an amount sufficient to cover the blade 1012 of the cutting tip 632, even when the blade 1012 is in its fully extended position. A clinician may for example place the outer jacket or guard 628 in this position such that if an accidental trigger press occurs, resulting in rotation and longitudinal translation of the cutting tip 632, the blade 1012 will nevertheless be protected from cutting tissues of the patient. In other instances, the shield may permit the blade to cut tissue while shielded, reducing potential to cut the vessel wall or lead. Tissue may be pulled into the cutting mechanism in this manner.

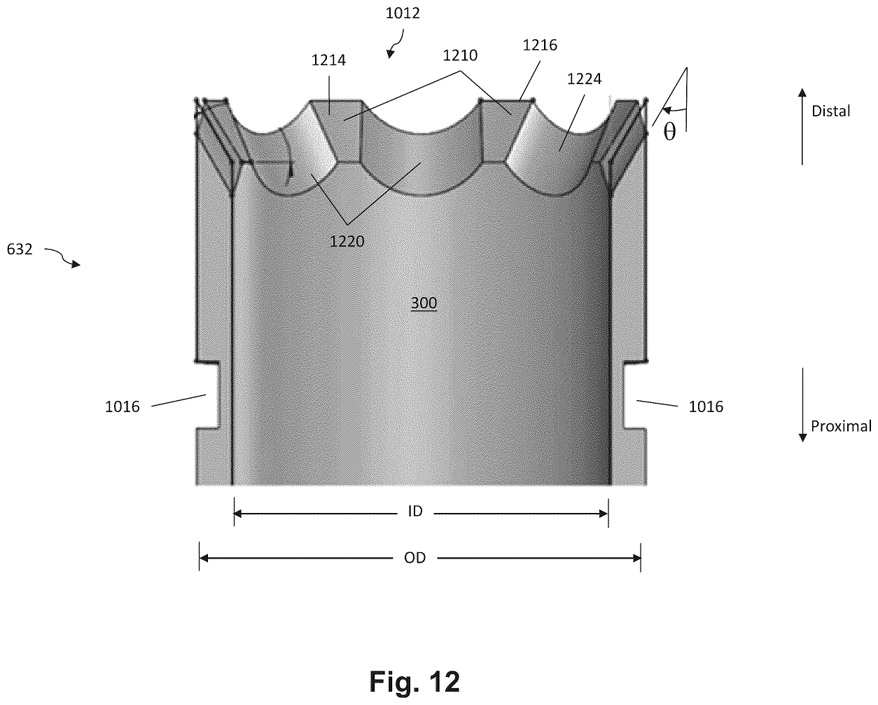

FIG. 12 is a side cross-sectional view of the cutting tip 632 of FIG. 8, according to aspects of the present disclosure. Visible are the cam slot 1016, inner lumen 300, and blade 1012. The cutting tip has an outer diameter OD and an inner diameter ID (e.g., the diameter of the inner lumen 300), as well as sidewall 1250, a proximal portion 1230, and a distal portion 1240 terminating at a distal end 1245. The blade includes a number of teeth 1210 and serrations 1220. Each tooth 1210 has a trapezoidal face 1214 chamfered inward toward the inner lumen 300, and a sharp distal edge 1216. Depending on the implementation, the length of the edges 1216 may be different than shown in FIG. 12. For example, the edges 1216 may be wider or narrower, or may form sharp points, or may be of more than one length. Each serration 1220 includes a curved surface 1224 (e.g., a hemicylindrical surface or other curved surface) chamfered inward toward the outer lumen. The chamfer of the teeth 1210 and serrations 1220 forms an angle of θ degrees with the vertical. In an example, θ is equal to −30 degrees, although other values both larger and smaller may be used instead or in addition.

The inward-facing chamfer (which may also be referred to as a negative chamfer) advantageously puts the sharp distal edges 1216 of the teeth 1210 as far as possible from the lead for a given outer diameter OD, such that as the lead passes into the inner lumen 300 of the cutting tip 632, the risk of accidentally cutting or snagging the lead is minimized.

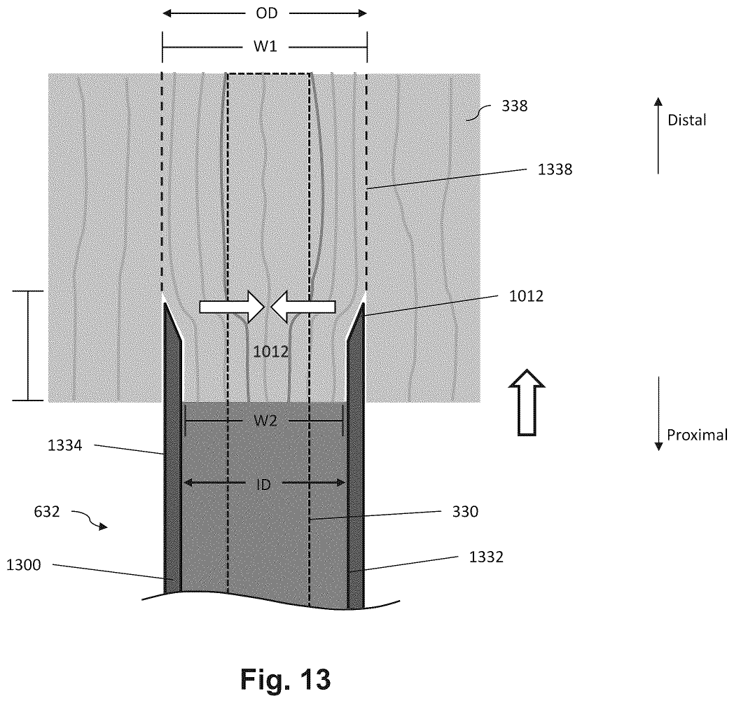

FIG. 13 is a schematic, diagrammatic, side cross-sectional view of an example cutting tip 632 cutting into tissue 338 surrounding a lead 330, according to aspects of the present disclosure. As the cutting tip 632 advances distally through the tissue 338, it cuts a cylindrical plug 1338 that includes both the lead 330 and a portion of the tissue 338 surrounding the lead 330. The tip of the blade 1012 is aligned with the outer wall surface 1334 of the wall 1300 of the cutting tip 632 (e.g., the outer surface of the wall 1250). Thus, at the tip of the blade 1212, the plug 1338 has a diameter or width W1, which is equal to the outer diameter OD of the cutting tip 632. However, because of the inward chamfer or negative chamfer of the blade 1012, as the plug 1338 moves proximally into the cutting tip 632, the plug must compress to a smaller diameter or width W2, which is equal to the inner diameter ID of the cutting tip 632. Because the lead 330 is relatively incompressible as compared with the tissue 338, the compression of the plug 1338 may be considered primarily a compression of the tissue component of the plug 1338. This compression creates an outward force between the plug 1338 and the inner wall surface 1332 of the wall 1300 of the cutting tip 632 (e.g., the inner surface of the wall 1250), thus leading to friction.

It is understood that a second shaft or outer sheath 624 and/or a third shaft or outer jacket 628, although not shown in FIG. 13, can also be provided (e.g., as shown in FIGS. 9-11).

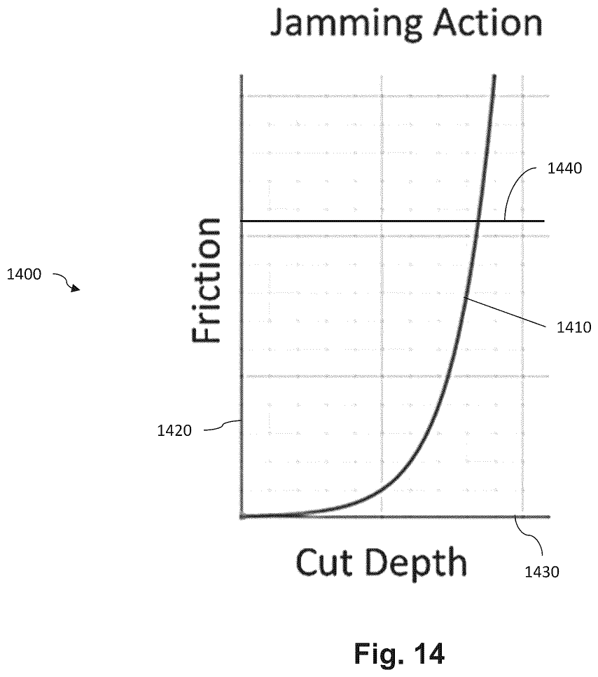

FIG. 14 is a graph 1400 showing a curve 1410 of compressive friction force 1420 as a function of the cut depth 1430 of the cutting tip into the tissue surrounding the lead, according to aspects of the present disclosure. The compressive friction force 1420 is the component of total friction that is caused by compression of the tissue inside the cutting tip. It is understood that other contributions to the total friction force may exist. As can be seen in the graph 1400, the compressive friction force 1420 increases exponentially with cut depth 1430 until it exceeds a jamming threshold 1440. Once the jamming threshold 1440 is exceeded, the cutting tip may jam within the tissue, making further distal advancement of the cutting tip into the tissue increasingly difficult, or even impossible, without risk to surrounding tissues such as the wall of the heart. Removal of the cutting catheter may also become more difficult once the cutting tip has jammed. Thus, a need exists for improved cutting tips that generate less compressive friction or no compressive friction and are thus less prone to jamming.

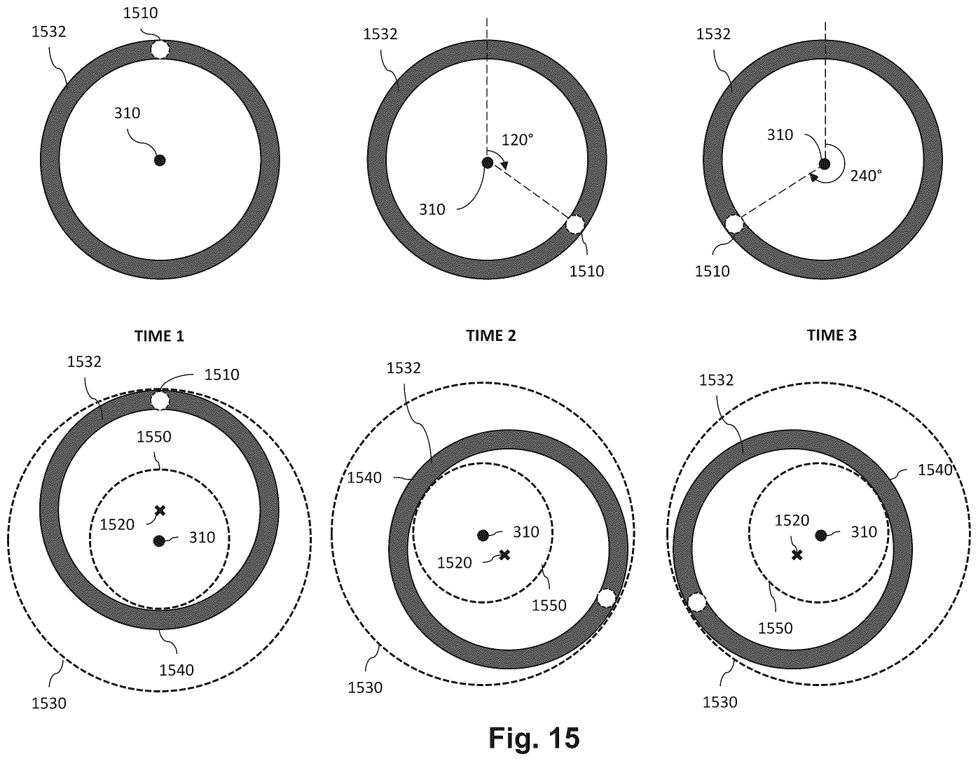

FIG. 15 is an end view of an example cutting tip 1532 rotating along an oscillating cut path, according to aspects of the present disclosure. With regard to the top row, showing rotational motion around longitudinal axis or central longitudinal axis 310 of the lead removal device, flexible elongate member, and/or cutting tip, at time 1, a reference point 1510 on the cutting tip (e.g., the location where the guide pin is fixedly coupled to the cutting tip) is at a first location. At time two, the reference point 1510 has rotated to a second location, approximately 120 degrees away from the first location. At time 3, the reference point 1510 has rotated to a third location, approximately 240 degrees from the first location.

However, in the example shown in FIG. 15, the guide pin is coupled to a cam slot in the outer band, which is larger than the cutting tip 1532, as shown and described below. Thus, as shown in the bottom row, the center 1520 of the cutting tip 1532 is moving from one time to the next, while the reference point 1510 (e.g., the guide pin 640) traces out an outer circumference 1530, which is equal to the inner diameter of the outer band. At the same time, a point 1540 directly opposite the reference point 1510 traces out an inner diameter 1550.

Thus, the rotational motion of the cutting tip 1532 (e.g., around the longitudinal axis 310) also generates a lateral or transverse motion (up-down and left-right with respect to the plane 1622 of the page, which is a plane perpendicular/orthogonal to the longitudinal axis 310). The difference between the inner diameter 1550 and outer diameter 1530 is the cut width generated by the cutting tip 1532, as described below.

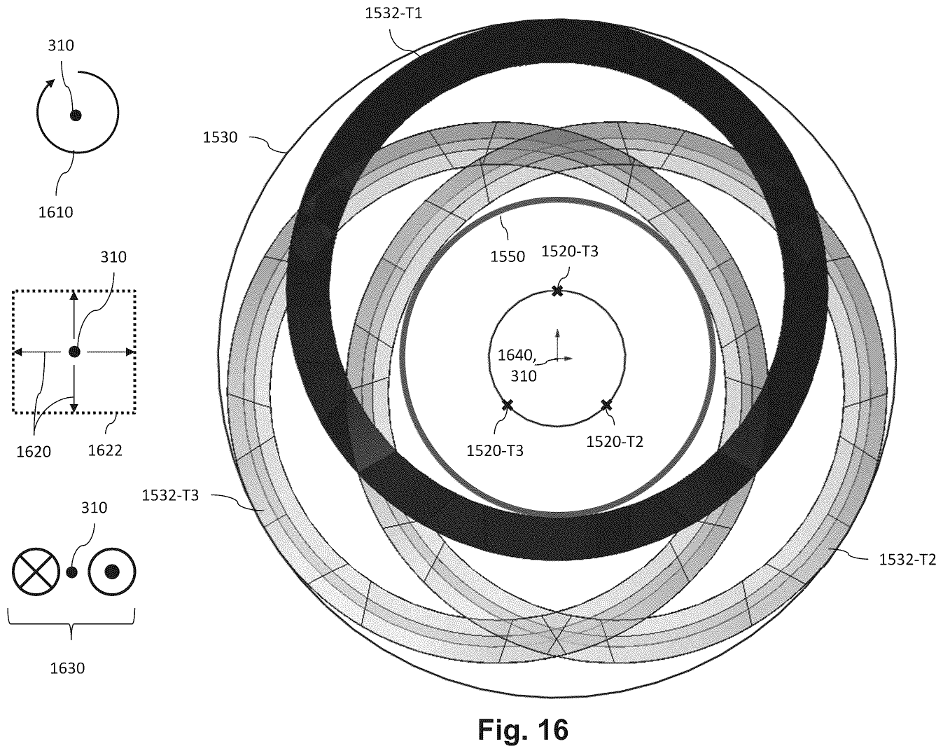

FIG. 16 is an end view of an example cutting tip 1532 rotating along an oscillating cut path, according to aspects of the present disclosure. Visible are the outer diameter 1530 (e.g., inner diameter of the outer band), inner diameter 1550, cutting tip 1532 at time 1, cutting tip 1532 at time 2, cutting tip 1532 at time 3, axial center point 1520 at time 1, axial center point 1520 at time 2, and axial center point 1520 at time 3. Thus, the cutting tip 1532 engages in both rotary or rotational motion 1610 (e.g., around the longitudinal axis 310 of the lead removal device, flexible elongate member, and/or cutting tip) and lateral or transverse motion 1620 (left/right and up/down in the plane 1622 of the page, which is a plane perpendicular to the longitudinal axis 310). Depending on the shape of the cam path, the cutting tip may also engage in longitudinal motion 1630 (e.g., proximal/distal, or into and out of the plane 1622 of the page, which is along the longitudinal axis 310). Also visible is the center of oscillation 1640, which can also be referenced as a central longitudinal axis or longitudinal axis 310.

It is noted that the cutting device described in FIGS. 1-14 may only have rotational motion and longitudinal motion; the device described by FIGS. 15-38 advantageously adds the lateral/transverse motion to increase the width of the cut and thus reduce the size of the plug.

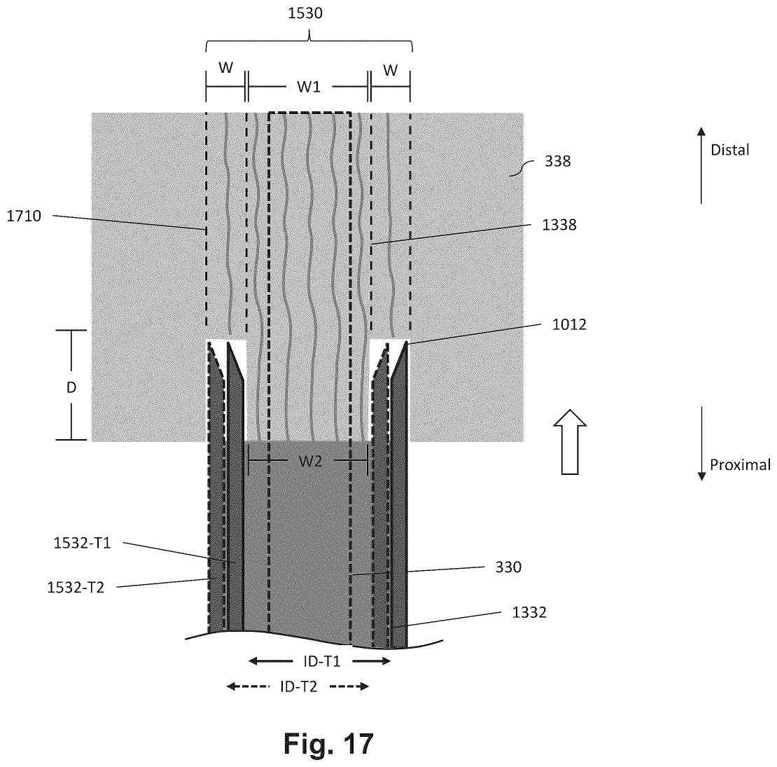

FIG. 17 is a schematic, diagrammatic, side cross-sectional view of an example oscillating cutting tip 1532 cutting into tissue 338 surrounding a lead 330, according to aspects of the present disclosure. The cutting tip 1532 is visible at a first position at time T1 and at a second position at time T2. Because of the spiral motion shown in FIG. 15-16, as the oscillating cutting tip 1532 advances distally through the tissue 338, it cuts a cylindrical plug 1338 that includes both the lead 330 and a portion of the tissue 338 surrounding the lead 330. At the tip of the blade 1012, the plug 1338 has a diameter or width W1, which is equal to the inner diameter 1550 of the cutting tip rotation (see FIG. 16), and smaller than the inner diameter ID of the cutting tip 1532. As the cutting tip traces out the outer diameter 1530, it creates a ring of shredded tissue 1710 with a width W that is equal to the outer diameter 1530 minus the inner diameter 1550 or W1. Some of the shredded tissue may remain in the vessel, while some may enter the lumen and/or be extracted along with the lead. The width W2 of the plug 1338 inside the cutting tip 1532 is equal to the width W1 of the plug 1338 at the tip of the blade 1012.

In an example, the distal end of the inner sheath is coupled to the proximal end of the cutting tip, and the inner diameter of the inner sheath is greater than the inner diameter of the cutting tip, and thus there is also little or no compression at or proximal of the transition between the cutting tip and the inner sheath. It is understood that a second shaft or outer sheath 624 and/or a third shaft or outer jacket 628, although not shown in FIG. 17, can also be provided (e.g., as shown in FIGS. 9-11).

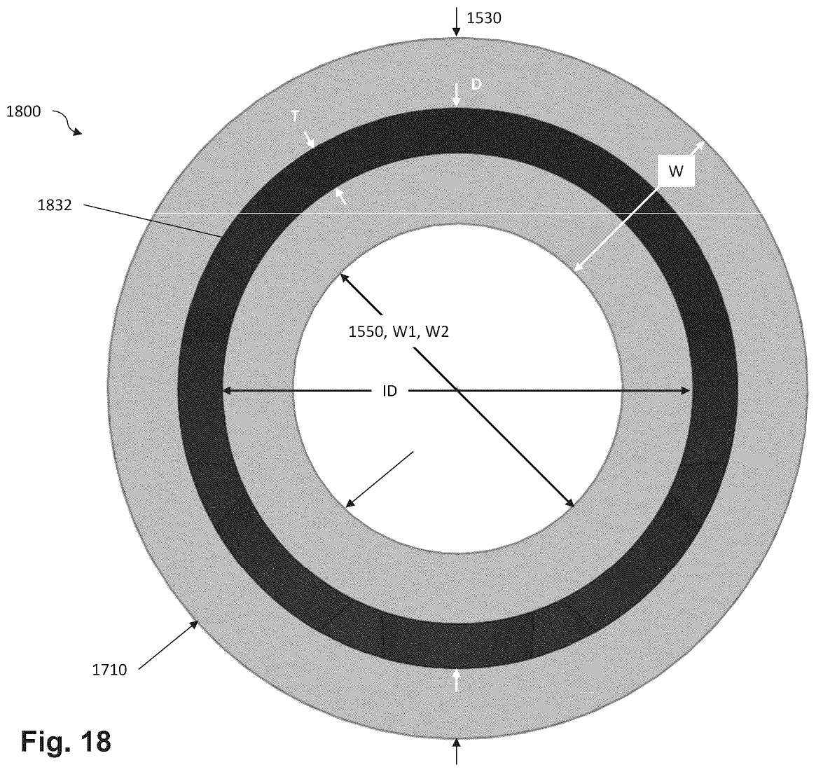

FIG. 18 is a schematic, diagrammatic end view of an example cut region 1800 created by an oscillating cut path, according to aspects of the present disclosure. Visible is the region 1832 that would be cut by a non-oscillating cutting blade of thickness T, diameter D, and inner diameter ID. Also visible is the ring-shaped shredded region 1710 created by the oscillating blade. The shredded region 1710 has width W, outer diameter 1530 (which, in the case of an inward-chamfered blade, is equal to the inner diameter of the outer band) and inner diameter 1550 (which is equal to widths W1 and W2 of FIG. 17). The tissue plug 1338 (see FIG. 17) also has a diameter of 1550, W1, or W2, such that it fits easily inside the inner diameter ID if the cutting tip 1532.

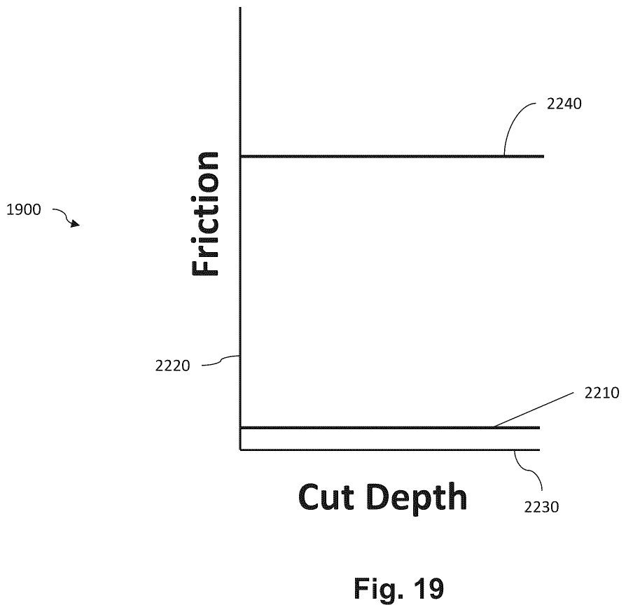

FIG. 19 is a graph 1900 showing a curve 1910 of compressive friction force 1920 as a function of the cut depth 1930 of the oscillating cutting tip into the tissue surrounding the lead, according to aspects of the present disclosure. As can be seen in the graph 1900, the compressive friction force 1920 has a small or even zero value that does not substantially increase with cut depth 1930 and does not approach the jamming threshold 1940. Thus, the oscillating cutting tip can be seen as substantially less likely to jam than the inward-chamfered cutting tip of FIG. 14.

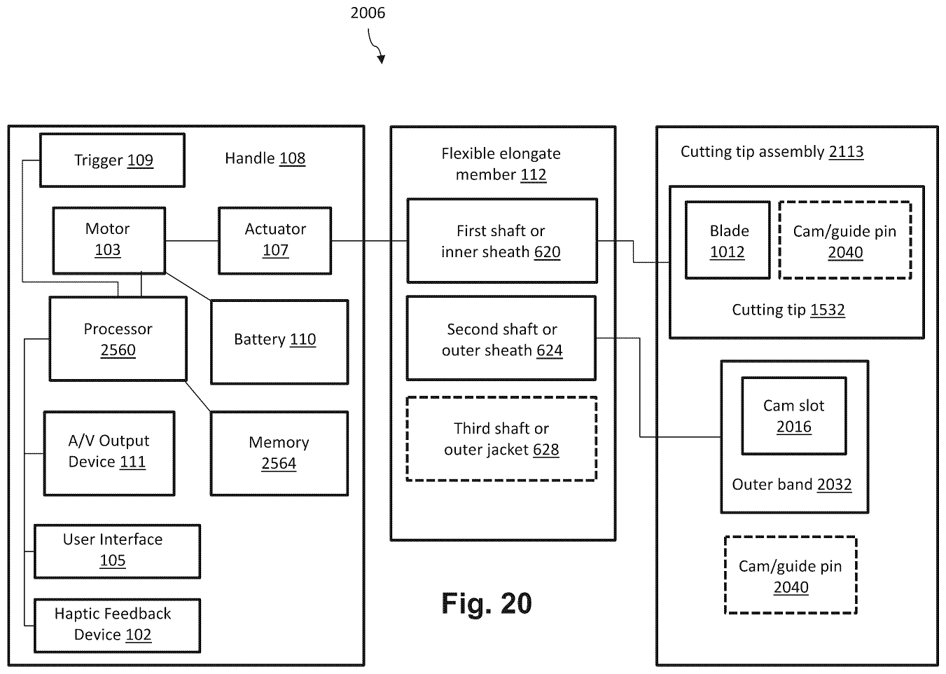

FIG. 20 is a diagrammatic schematic view of an intraluminal cutting device or surgical device 2006, such as a cutting catheter device, according to aspects of the present disclosure. The intraluminal cutting device or surgical device 2006 includes a handle 108, a flexible elongate member or sheath assembly 112, and a cutting tip assembly 2113.

The handle 108 may include a trigger 109, one or more motors 103, an actuator 107, a processor 2560, a battery 110, an audiovisual (A/V) output device 111 (e.g., a display screen, a set of indicator lights, etc.), and a user interface 105 (e.g., a touchscreen, one or more buttons, switches, dials, etc.).

The flexible elongate member or sheath assembly 112 may include a rotatable first shaft or flexible inner sheath 620 coupled to the actuator 107, a fixed second shaft or flexible outer sheath 624 that surrounds the flexible inner sheath 620, and a translatable third shaft or outer jacket 628 that surrounds the flexible outer sheath 624.

In an example, the actuator 107 be or include a power train assembly with one or more gears coupling the 103 motor to the first (inner) shaft or flexible inner sheath 620. A press of the trigger 109 may send a signal to the processor 2560, which then activates the motor 103, which is powered by the battery 110 and transmits rotational motion through the actuator 107 to the flexible inner sheath 620, which then rotates along with the actuator. The flexible outer sheath 624 may be fixed to the handle 108, such that it remains fixed while the flexible inner sheath 620 rotates within it.

The cutting tip assembly 2113 may for example include an outer band 2032 that includes a cam slot 2016, as well as a cutting tip 1532 with a guide pin 2040 and blade 1012. The cutting tip 1532 is rotatably attached to the cam slot 2016 of the outer band 2032, such that rotation of the flexible inner shaft 620 causes rotation of outer band 2032 and cutting tip 632. The cam pin, guide pin, or cam guide pin 2040 is fixedly attached to the cutting tip 1532, but fits within the cam slot 2016, such that rotation of the cutting tip 1532 can also drive axial or longitudinal motion of the cutting tip 1532, as described below.

The third shaft or outer jacket 628 surrounds the second shaft or outer sheath 624, and is translatably movable along it, such that a distal portion of the outer jacket 628 can be advanced to cover the blade 1012 of the cutting tip 632, as described above.

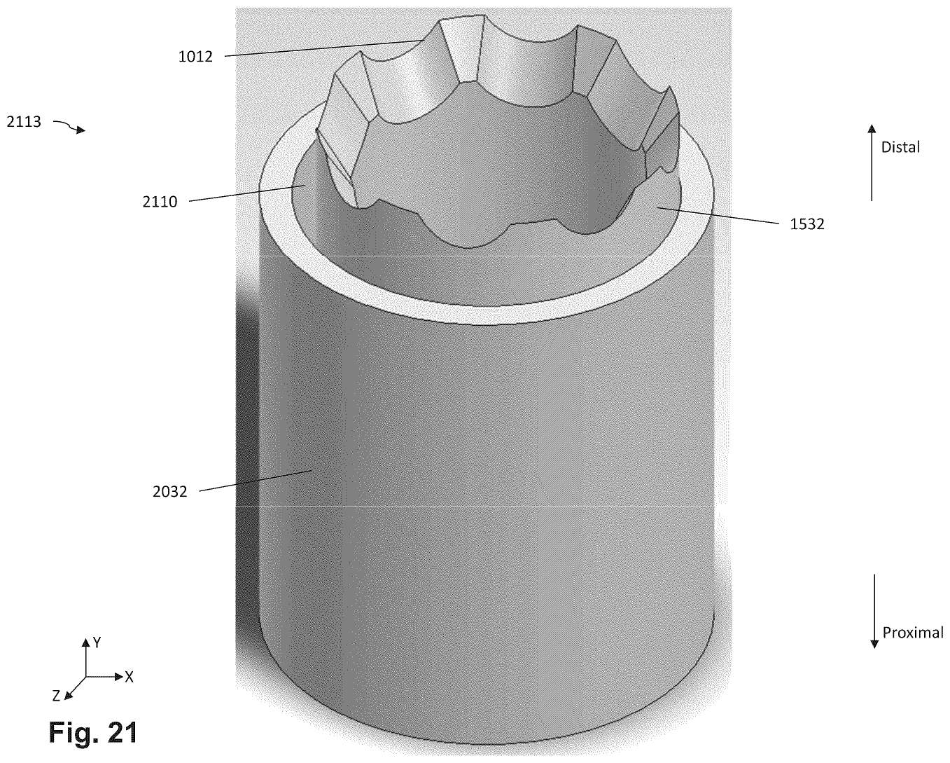

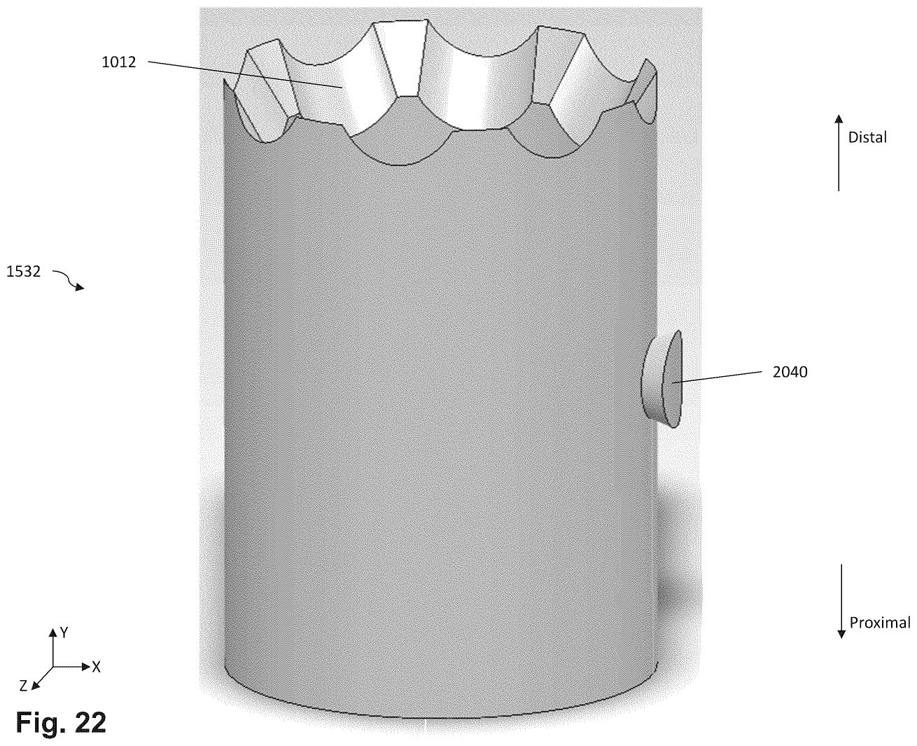

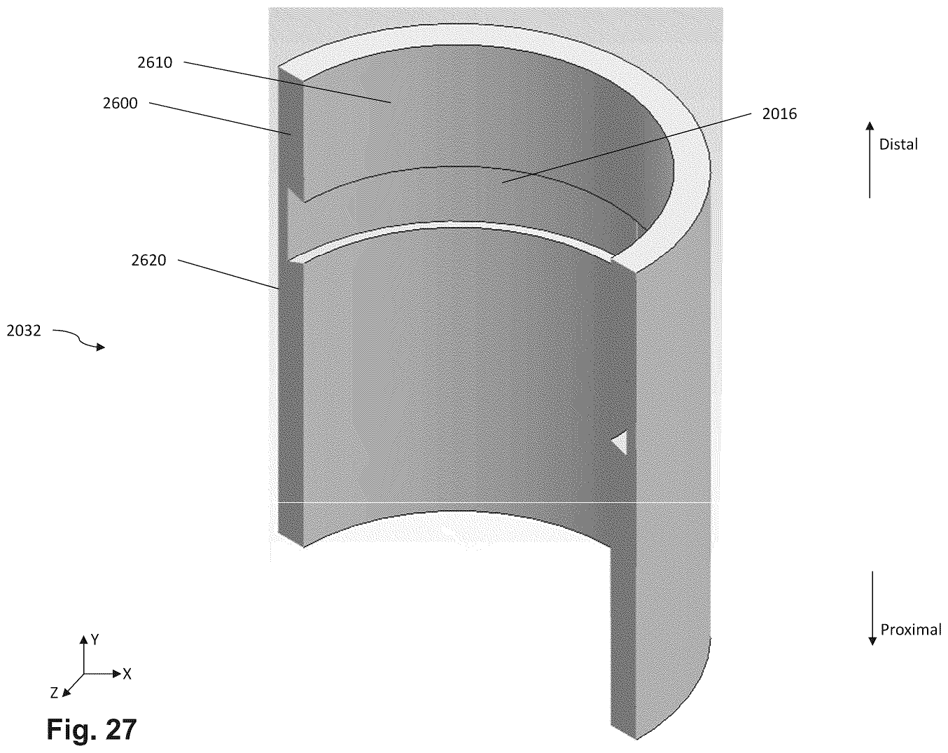

Thus, there are several differences between the cutting device or surgical device 2006 of FIG. 20 and the cutting device or surgical device 106 of FIG. 1, including:
1) Non-concentric rotation of the cutting tip 1532 (and also the inner sheath 620, which is attached to the cutting tip), as shown for example in FIGS. 15-16. The cutting device 106 of FIG. 1 has concentric rotation of the cutting tip 632 (and inner sheath 620).
2) This non-concentric rotation causes lateral movement of the cutting tip 1532 (and also of the inner sheath 620, attached to the cutting tip 1532), which occurs along with the longitudinal movement and rotational movement described above. The cutting device 106 of FIG. 1 may have only longitudinal movement and rotational movement of the cutting tip 632-not lateral movement.
3) The guide pin 2040 is shaped for positive retention within the cam slot 2016 (e.g., a spool-shaped or dovetail-shaped guide pin 2040, as shown below in FIGS. 22-24, 30-31, and 34-37), whereas the cutting device 106 of FIG. 1 may have a cylindrical guide pin 640 lacking this feature (see e.g., FIG. 3).
4) The guide pin 2040 is fixed to the outer surface of the cutting tip 1532, and the cam slot 2016 is on the inner surface of the outer band 2032, whereas for the cutting device 106 of FIG. 1, the guide pin 640 is fixed to the inner surface of the outer sheath 624, and the cam slot 1016 is on the outer surface of the cutting tip 632 (see e.g., FIGS. 3 and 8).
5) The cam slot 2016 is shaped for positive retention of the guide pin 2040 (e.g., a T-shaped or dovetail-shaped cross-section as shown below in FIGS. 27-30), whereas for the cutting device 106 of FIG. 1, the cam slot 1016 has a uniform (e.g., rectangular) cross-section not shaped for positive retention of the guide pin 640 (see e.g., FIG. 12).

Depending on the implementation, other differences may also exist between the cutting device 2006 of FIG. 20 and the cutting device 106 of FIG. 1, without departing from the spirit of the present disclosure.

The guide pin 2040 may be formed as part of the cutting tip 1532, or could be a separate component that is fixedly coupled to the cutting tip (e.g., by adhesive, solder, welding, etc.).

FIG. 21 is a top side perspective view of at least a portion of an example cutting tip assembly 2113, according to aspects of the present disclosure. Visible are the cutting tip 1532, blade or cutting edge 1012, and outer band 2032. Because of the offset rotation of the cutting tip 1532 within the outer band 2032, a gap 2110 exists on one side between the cutting tip 1532 and the outer band 2032. The location of the gap 2110 moves around the inner circumference of the outer band 2032 as the cutting tip 1532 rotates.

In the example shown in FIG. 21, the blade 1012 is configured with an inward chamfer. However, it is understood that the offset cutting mechanisms disclosed herein can also be used with a blade having an outward chamfer, a dual chamfer, no chamfer, and other blade designs, whether currently in existence or hereinafter developed.

FIG. 22 is a top side perspective view of an example cutting tip 1532, according to aspects of the present disclosure. Visible are the blade or cutting edge 1012 and the guide pin 2040. The guide pin 2040 has a dovetail shape, for positive retention in the dovetail-shaped cam slot of the outer band (see FIGS. 27-30, below).

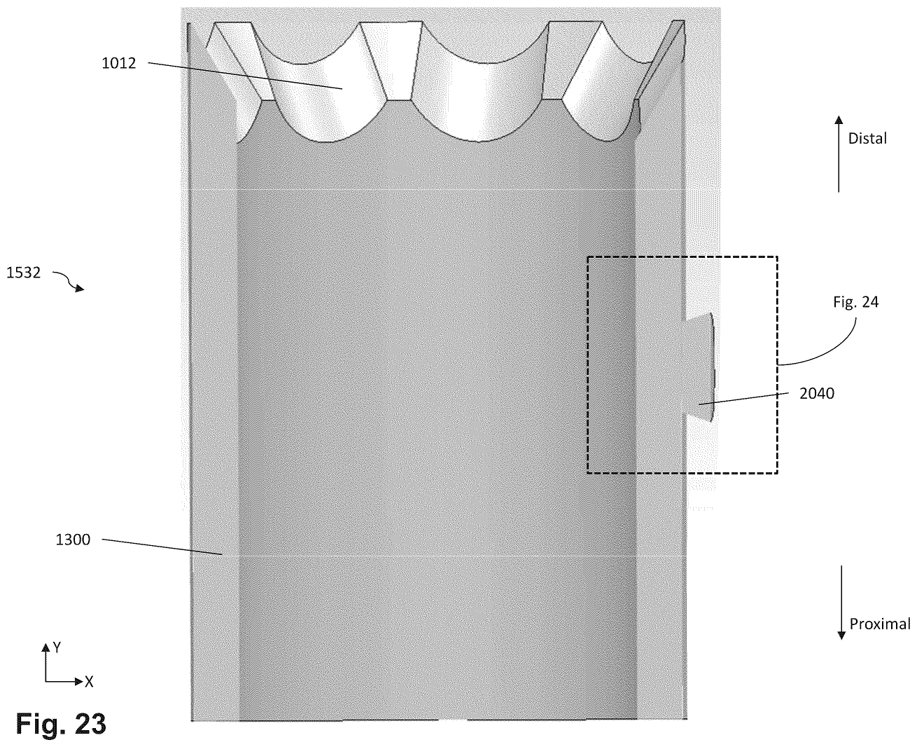

FIG. 23 is a lateral cross-sectional view of an example cutting tip 1532, according to aspects of the present disclosure. Visible are the blade or cutting edge 101, the guide pin 2040, and the wall 1300 of the cutting tip 1532. The guide pin 2040 has a dovetail shape, for positive retention in the dovetail-shaped cam slot of the outer band (see FIGS. 27-30, below).

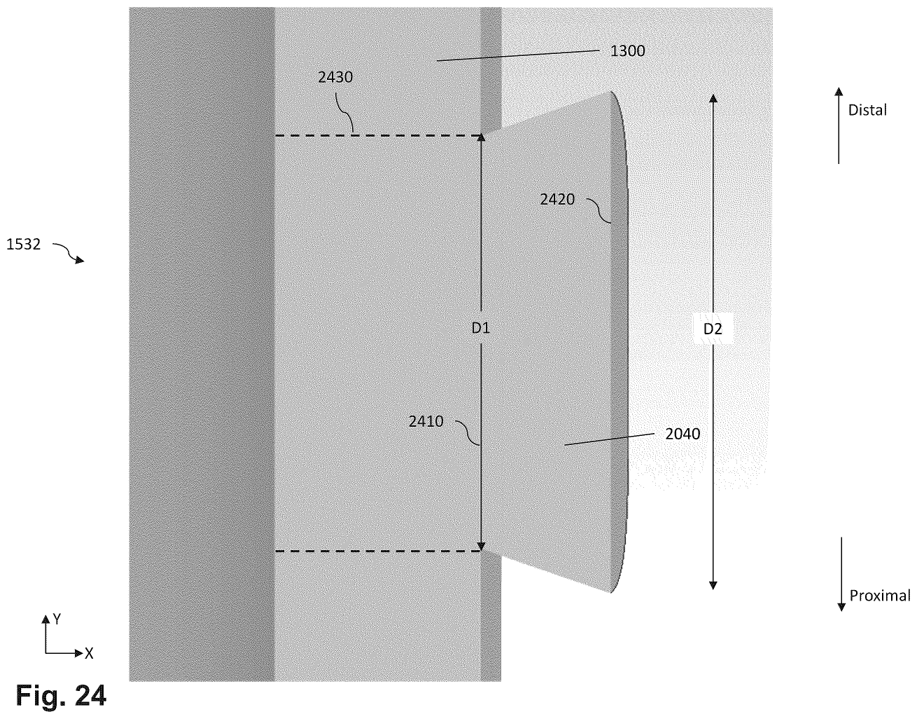

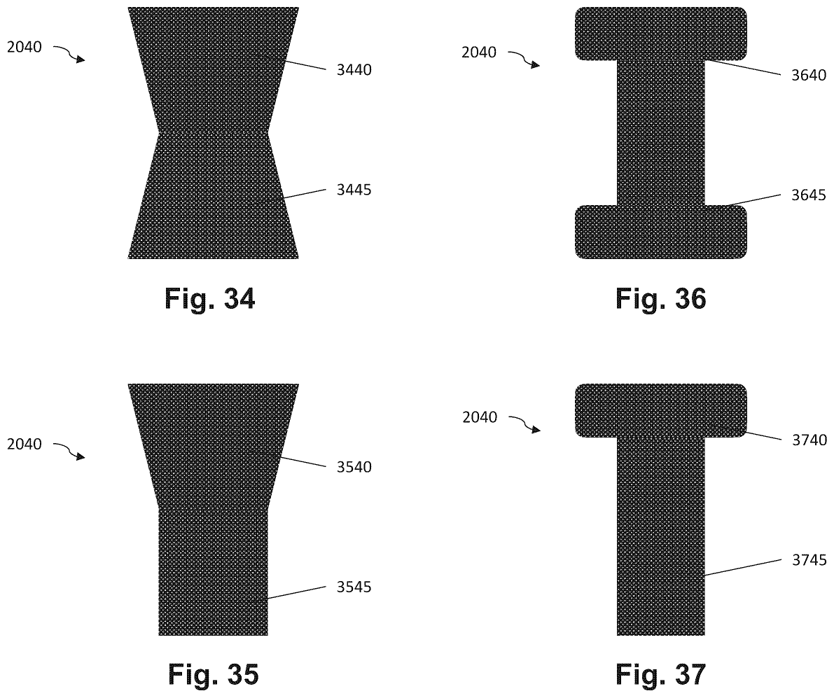

FIG. 24 is an enlarged view of the guide pin 2040 of FIG. 23, according to aspects of the present disclosure. To facilitate positive retention by the dovetail-shaped cam slot on the inner surface of the outer band, the guide pin 2040 may be dovetail shaped. It is noted that other shapes for the guide pin and cam slot are possible and fall within the scope of the present disclosure, as shown below for example in FIGS. 34-37. In an example, the guide pin 2040 is circular frustum shaped, with a diameter D1 at its inner end 2410 (e.g., at the wall 1300 of the cutting tip 1532), and a larger diameter D2 at its outer end 2420.

Depending on the implementation, the guide pin 2040 may be formed (e.g., cast, printed, or machined) as part of the wall 1300 of the cutting tip 1532, or may be s separate piece that may for example be inserted into a through-hole 2430 and attached (e.g., glued, soldered, welded, etc.) to the wall 1300.

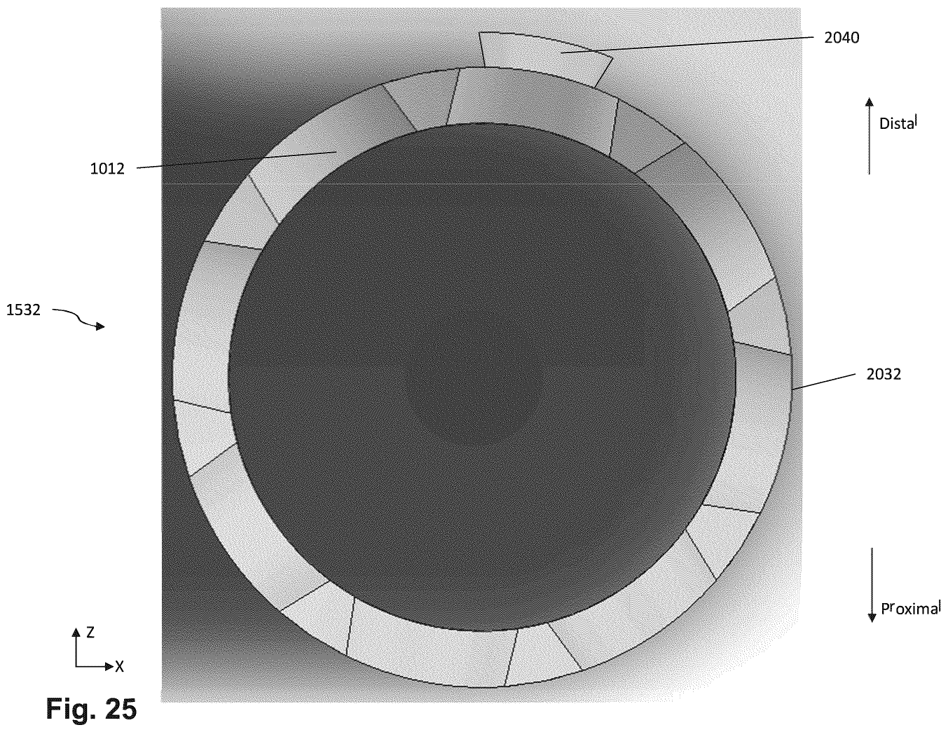

FIG. 25 is an end view of an example cutting tip 1532, according to aspects of the present disclosure. Visible are the wall 1300, blade or cutting edge 1012, and guide pin 2040. As the guide pin 2040 slides through the cam slot in the inner surface of the outer band, the cutting tip 1532 rotates in an offset manner as shown above in FIGS. 15 and 16.

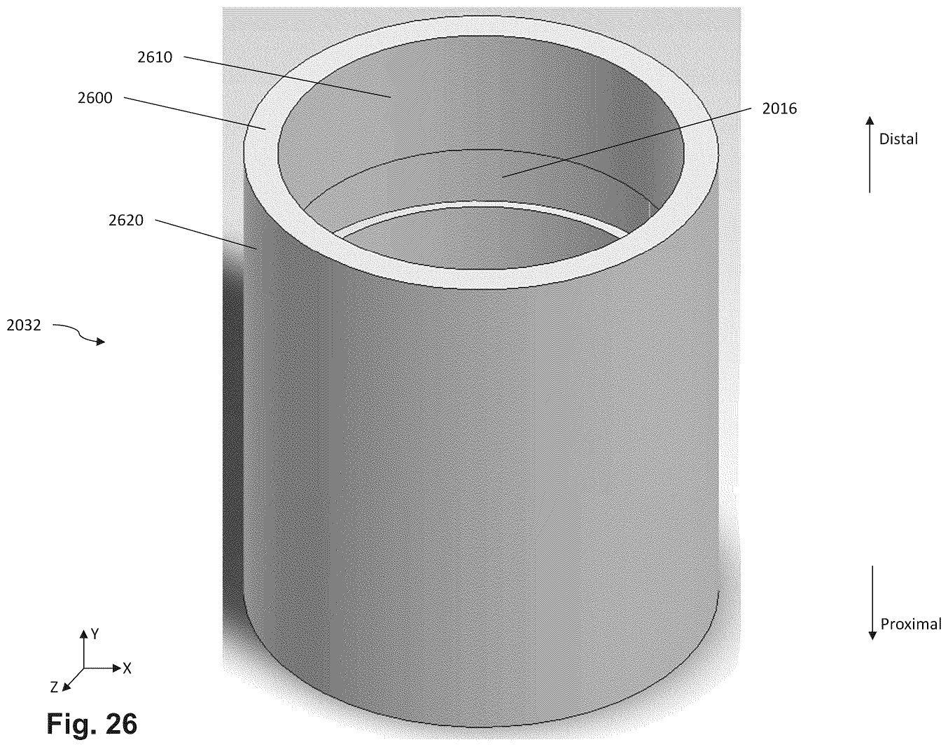

FIG. 26 is a top side perspective view of an example outer band 2032, according to aspects of the present disclosure. The outer band includes a wall 2600 with an inner surface 2610 and outer surface 2620. The cam slot 2016 can be formed (e.g., cast, printed, or machined) into the inner surface 2610 of the wall 2600.

FIG. 27 is a top side perspective cross-sectional view of an example outer band 2032, according to aspects of the present disclosure. Visible are the wall 2600, inner surface 2610, outer surface 2620, and cam slot 2016. For positive retention of the guide pin, the cam slot 2016 can be formed (e.g., cast, printed, or machined) into the inner surface 2610 of the wall 2600 in a dovetail shape, although other shapes (e.g., T-shaped) may be used instead or in addition.

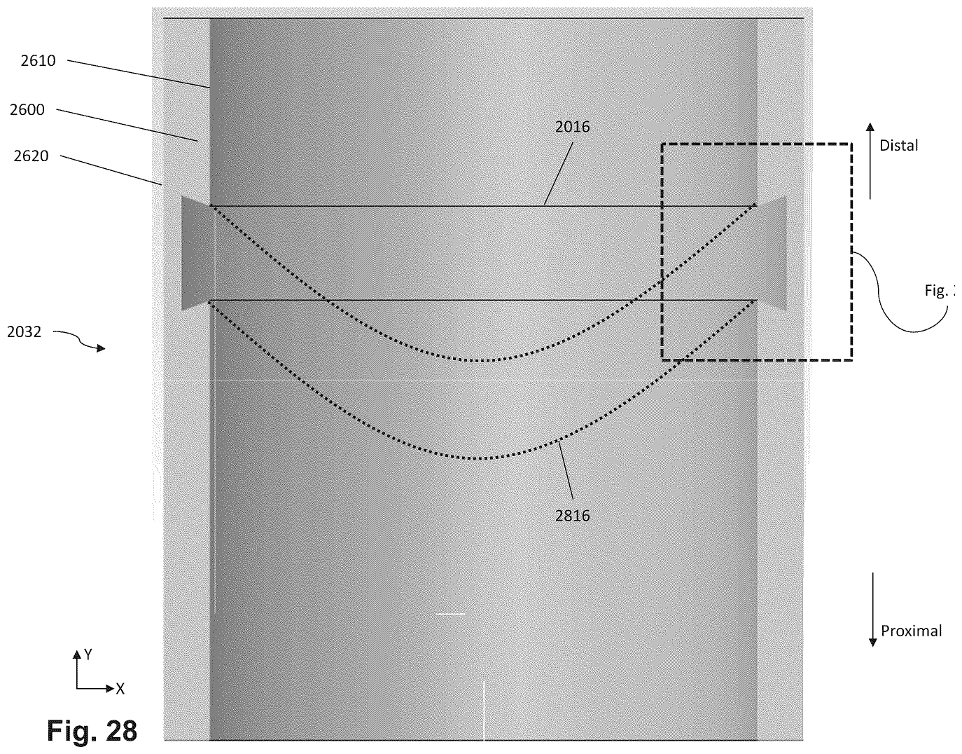

FIG. 28 is a lateral cross-sectional view of an example outer band 2032, according to aspects of the present disclosure. Visible are the wall 2600, inner surface 2610, outer surface 2620, and cam slot 2016. In the example shown in FIG. 28, for positive retention of the guide pin, the cam slot 2016 has been formed (e.g., cast, printed, or machined) into the inner surface 2610 of the wall 2600 in a dovetail shape. It is noted that the straight cam slot 2016 shown in FIG. 28 will not result in longitudinal motion of the cutting tip as the cutting tip rotates within the outer band 2032. However, a curved cam slot 2816 would cause the guide pin to move longitudinally (e.g., proximally and distally) as it slides through the cam slot 2816, and would thus result in longitudinal (e.g., proximal and distal) motion of the cutting tip, as well as the rotational and lateral motion described above in FIGS. 15 and 16 (in a plane 1622 perpendicular to the longitudinal axis 310).

FIG. 29 is an enlarged view of the cam slot 2016 of FIG. 28, according to aspects of the present disclosure. In the example shown in FIG. 29, in order to facilitate retention of the guide pin as the cutting tip rotates within the outer band 2032, the dovetail-shaped cam slot 2016 has an inner width D3 which is equal to or slightly larger than the diameter D1 of the guide pin 2040 of FIG. 24, and a larger outer width D4 which is equal to or slightly larger than the diameter D2 of the guide pin 2040 of FIG. 24.

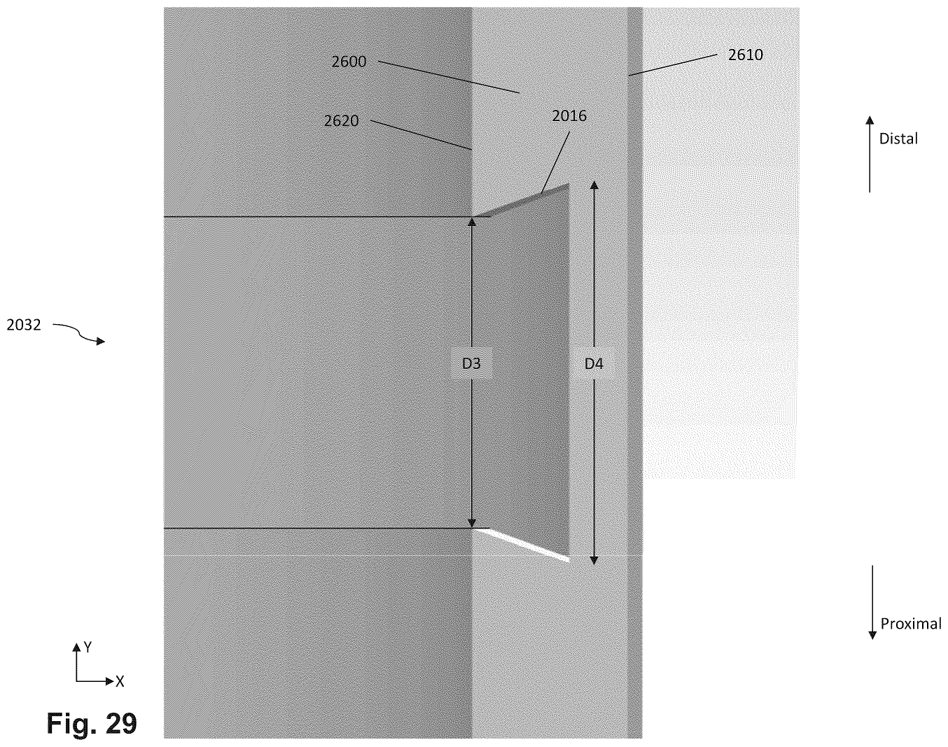

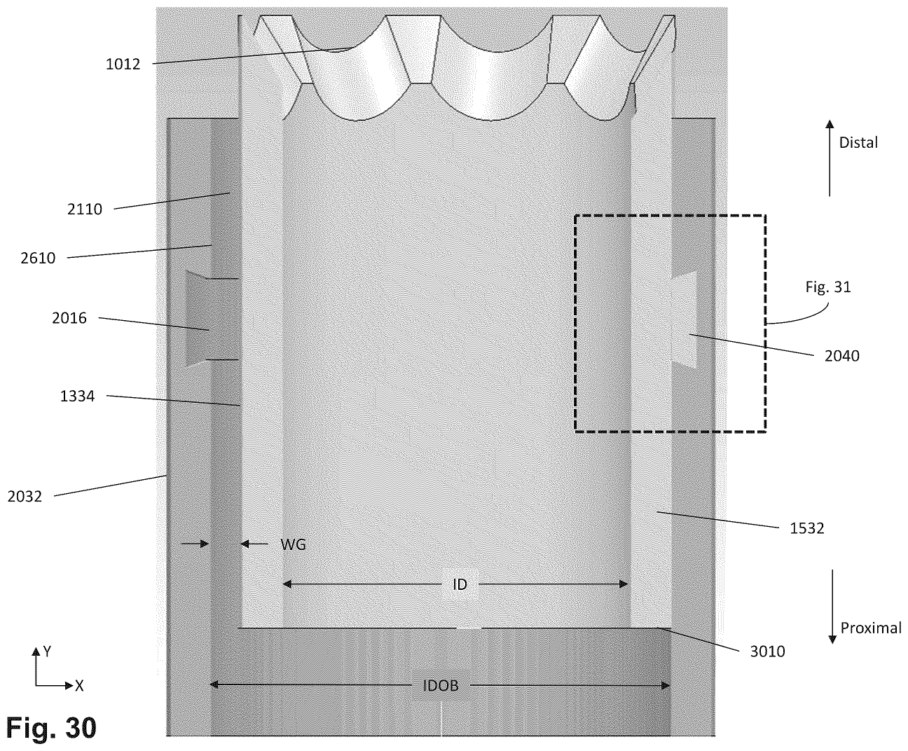

FIG. 30 is a lateral cross-sectional view of a cutting tip 1532 positioned within an example outer band 2032, according to aspects of the present disclosure. Visible are the blade 1012, guide pin 2040, cam slot 2016, and the gap 2110 between the outer surface 1334 of the cutting tip 1532 and the inner surface 2610 of the outer band 2032. The gap 2110 is widest at a point opposite (e.g., 180 degrees away from) the guide pin 2040, and is narrowest (e.g., approximately zero) at the guide pin 2040. The width WG of the gap 2110 determines the amount of offset motion or lateral motion the cutting tip 1532 will undergo over the course of a rotation within the outer band 1032. The inner diameter IDOB of the outer band 2032 is less than the inner diameter ID of the cutting tip 1532, and significantly less than the width W1 of the cut tissue plug 1338 (see FIG. 17). The inner sheath 620 attaches to, and rotates/translates with, the distal end 3010 of the cutting tip 1532 (see e.g., FIG. 9), and has the same or a similar inner diameter to that of the cutting tip 1532, such that the plug 1338 moves into and through the inner sheath 620 without being compressed.

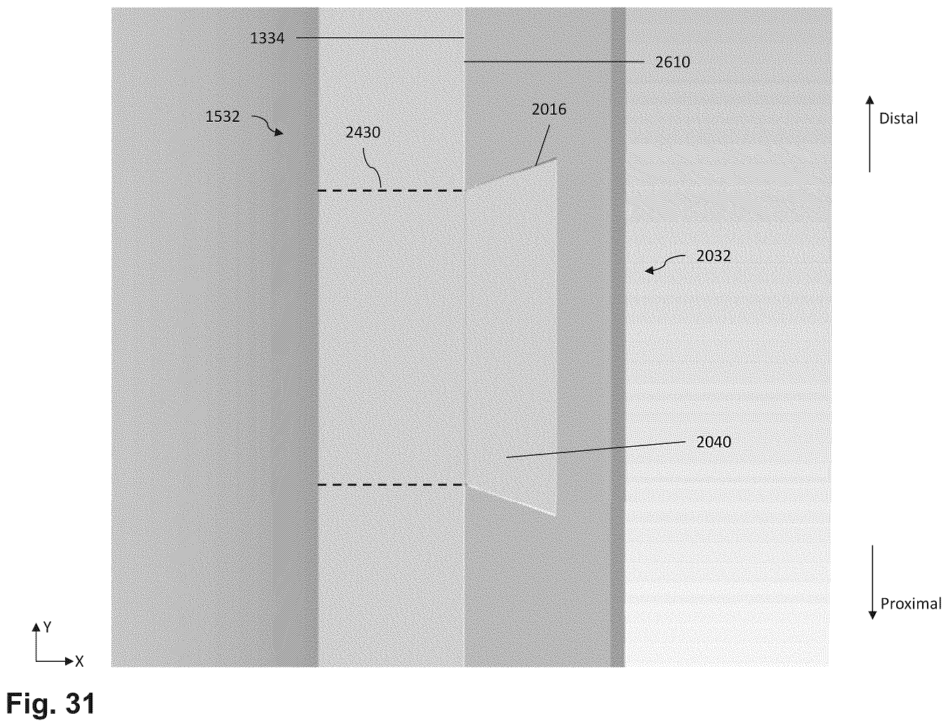

FIG. 31 is an enlarged view of the guide pin 2040 and cam slot 2016 of FIG. 30, according to aspects of the present disclosure. Visible is the though-hole 2430. In the example shown in FIG. 31, the dovetail-shaped guide pin 2040 and cam slot 2016 are sized and shaped such that the guide pin 2040 fits snugly, but slidably, into the cam slot 2016, such that the inner surface 2610 of the outer band is in sliding contact with the outer surface 1334 of the cutting tip 1532 in the vicinity of the guide pin 2040.

FIG. 32 is an end view of an example cutting tip 1532 positioned within an example outer band 2032, according to aspects of the present disclosure. Visible is the gap 2110 between the inner surface 2610 of the outer band 2032 and the outer surface 1334 of the cutting tip 1532. The cutting tip 1532 can be eccentrically or non-concentrically positioned relative to the outer band 2032 through the cutting tip's entire range of rotational motion and/or lateral motion (as shown in, e.g., FIGS. 15 and 16).

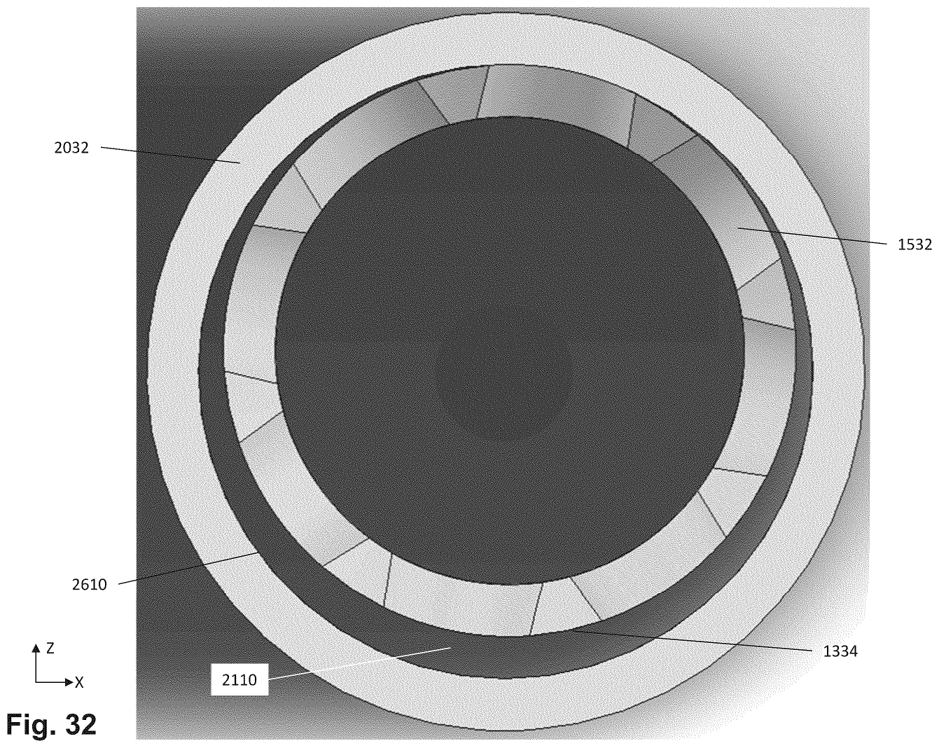

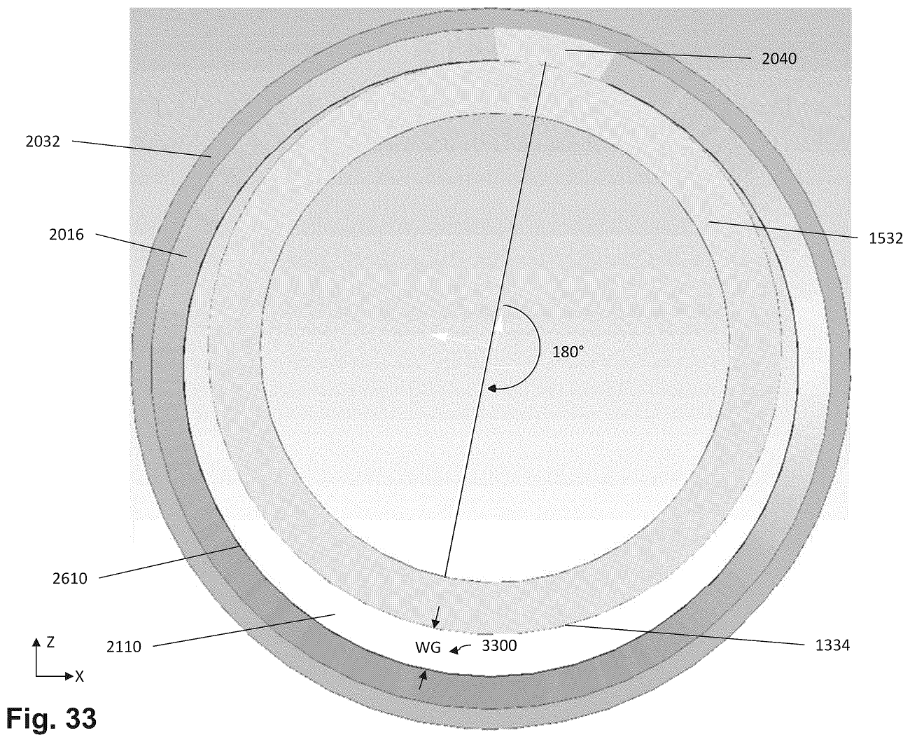

FIG. 33 is an end cross-sectional view of an example cutting tip 1532 positioned within an example outer band 2032, according to aspects of the present disclosure. Visible are the guide pin 2040, cam slot 2016, and the gap 2110 between the inner surface 2610 of the outer band 2032 and the outer surface 1334 of the cutting tip 1532. Proximate to the guide pin 2040, the width of the gap 2110 is approximately zero. The width of the gap 2110 is at its maximum value WG at a point 3300 that is directly opposite (e.g., 180 degrees away from) the guide pin 2040.

FIG. 34 is a cross-sectional view of an example guide pin 2040, according to aspects of the present disclosure. In the example shown in FIG. 34, the guide pin 2040 includes a first dovetail-shaped portion 3440 configured to fit into and be retained within the cam slot of the outer band (e.g., to prevent separation of the cam pin from the cam path), and a second dovetail-shaped portion 3445 configured to fit into and be retained by a through-hole in the wall of the cutting tip.

FIG. 35 is a cross-sectional view of an example guide pin 2040, according to aspects of the present disclosure. In the example shown in FIG. 35, the guide pin 2040 includes a dovetail-shaped portion 3540 configured to fit into and be retained within the cam slot of the outer band, and a cylindrical portion 3545 configured to fit into and be attached (e.g., glued, soldered, welded, etc.) to a through-hole in the wall of the cutting tip. The example guide pin 2040 of FIG. 35 is similar to the guide pin 2040 shown in FIG. 23. FIG. 36 is a cross-sectional view of an example spool-shaped guide pin 2040,

according to aspects of the present disclosure. In the example shown in FIG. 34, the guide pin 2040 includes a first T-shaped portion 3640 configured to fit into and be retained within the cam slot of the outer band, and a second T-shaped portion 3645 configured to fit into and be retained by a through-hole in the wall of the cutting tip.

FIG. 37 is a cross-sectional view of an example guide pin 2040, according to aspects of the present disclosure. In the example shown in FIG. 34, the guide pin 2040 includes a T-shaped portion 3740 configured to fit into and be retained within the cam slot of the outer band, and a cylindrical portion 3445 configured to fit into and be attached (e.g., glued, soldered, welded) to a through-hole in the wall of the cutting tip.

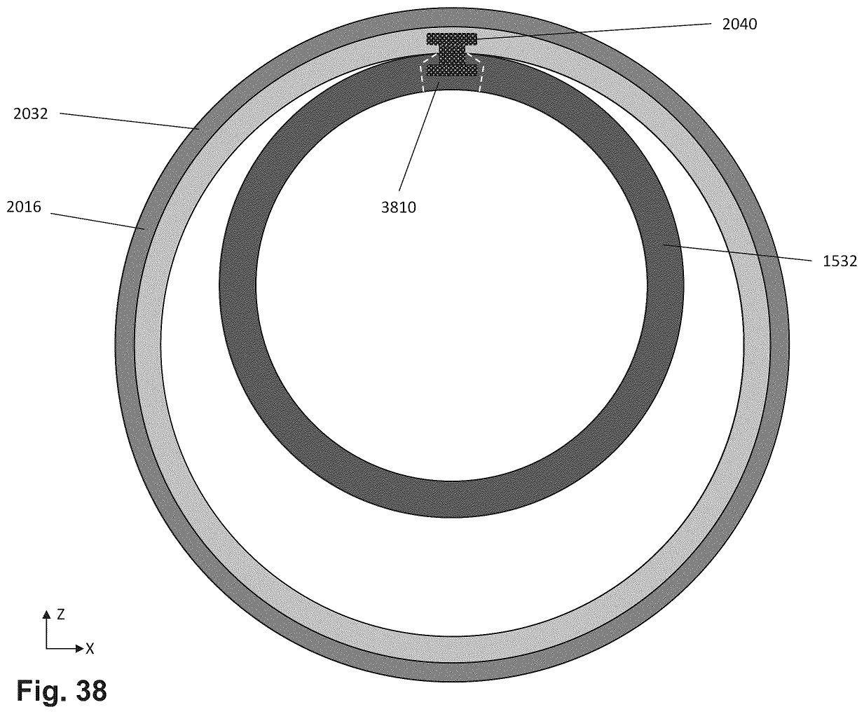

FIG. 38 is an end cross-sectional view of an example cutting tip 1532 inside an example outer band, according to aspects of the present disclosure. Visible are the guide pin 2040 and cam slot 2016. In the example shown in FIG. 38, the guide pin 2040 is spool-shaped (similar to the guide pin 2040 of FIG. 36), and the cam slot 2016 is sized and shaped to slidably retain the spool-shaped guide pin 2040. The cutting tip 1532 includes an attachment mechanism 3810 (e.g., one or more of a through-hole, a slot, a tongue-and-groove fitting, an adhesive-filled recess, a weld, etc.) to fixedly retain the guide pin 2040.

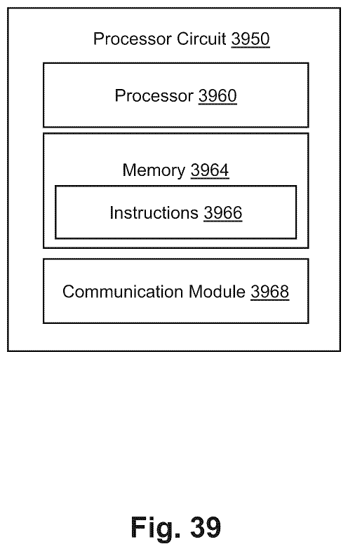

FIG. 39 is a schematic diagram of a processor circuit 3950, according to aspects of the present disclosure. The processor circuit 3950 may be implemented in the intraluminal cutting device 106, the intraluminal cutting device 2006, or other devices or workstations (e.g., third-party workstations, network routers, etc.), or in a cloud processor or other remote processing unit, as necessary to implement the method. As shown, the processor circuit 3950 may include a processor 3960, a memory 3964, and a communication module 3968. These elements may be in direct or indirect communication with each other, for example via one or more buses.

The processor 3960 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 3960 may also comprise another hardware device, a firmware device, or any combination thereof configured to perform the operations described herein. The processor 3960 may also be implemented as a combination of computing devices, e.g., a combination of a DSP and a microprocessor, a plurality of microprocessors, one or more microprocessors in conjunction with a DSP core, or any other such configuration.

The memory 3964 may include a cache memory (e.g., a cache memory of the processor 3960), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an aspect, the memory 3964 includes a non-transitory computer-readable medium. The memory 3964 may store instructions 3966. The instructions 3966 may include instructions that, when executed by the processor 3960, cause the processor 3960 to perform the operations described herein. Instructions 3966 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 3968 can include any electronic circuitry and/or logic circuitry to facilitate direct or indirect communication of data between the processor circuit 3950, and other processors or devices. In that regard, the communication module 3968 can be an input/output (I/O) device. In some instances, the communication module 3968 facilitates direct or indirect communication between various elements of the processor circuit 3950 and/or the intraluminal cutting device 106. The communication module 3968 may communicate within the processor circuit 3950 through numerous methods or protocols. Serial communication protocols may include but are not limited to US SPI, I²C, RS-232, RS-485, CAN, Ethernet, ARINC 429, MODBUS, MIL-STD-1553, or any other suitable method or protocol. Parallel protocols include but are not limited to ISA, ATA, SCSI, PCI, IEEE-488, IEEE-1284, and other suitable protocols. Where appropriate, serial and parallel communications may be bridged by a UART, USART, or other appropriate subsystem.

External communication (including but not limited to software updates, firmware updates, preset sharing between the processor and central server, or readings from the surgical device) may be accomplished using any suitable wireless or wired communication technology, such as a cable interface such as a USB, micro USB, Lightning, or Fire Wire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media 610 such as a USB flash drive or memory stick.

A number of variations are possible on the examples and aspects described above. For example, the technology described herein may be applied to cutting catheters and cutting tip blades of diverse types, whether currently in existence or hereinafter developed. All aspects described herein could be used in existing or novel medical procedures, including but not limited to lead extraction procedures. The principles described herein could be utilized in almost any industry that uses a round rotating cutter, including but not limited to core sampling drills. The cutting tip designs described herein can be detected by visual inspection.

Accordingly, the logical operations making up the aspects of the technology described herein are referred to variously as operations, steps, objects, elements, components, or modules. Furthermore, it should be understood that these may occur or be performed or arranged in any order, unless explicitly claimed otherwise or a specific order is inherently necessitated by the claim language. All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the intraluminal cutting device. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” Unless otherwise noted in the claims, stated values shall be interpreted as illustrative only and shall not be taken to be limiting.

The above specification, examples and data provide a complete description of the structure and use of exemplary aspects of the intraluminal cutting device as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter. Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. An apparatus, comprising:
a lead removal device configured to be positioned within a blood vessel of a patient and comprising:
a longitudinal axis;
a first flexible elongate sheath extending along the longitudinal axis and comprising a cutting tip configured to cut tissue to separate a medical lead implanted in the tissue; and
a second flexible elongate sheath extending along the longitudinal axis, wherein the first flexible elongate sheath is positioned inside the second flexible elongate sheath,
wherein the cutting tip is configured to have movement relative to the second flexible elongate sheath, wherein the movement comprises lateral motion in a plane perpendicular to the longitudinal axis.

2. The apparatus of claim 1, wherein the movement comprises:
rotational motion around the longitudinal axis; and
longitudinal motion along the longitudinal axis.

3. The apparatus of claim 2, wherein the lateral motion of the cutting tip is configured to cause the rotational motion of the cutting tip to be non-concentric relative to the second flexible elongate sheath.

4. The apparatus of claim 1, wherein the lead removal device further comprises a cam pin comprising a first end fixedly coupled to the cutting tip.

5. The apparatus of claim 4,
wherein the second flexible elongate sheath comprises a band,
wherein the band comprises a cam path such that the cam path is positioned radially outward of the first end of the cam pin,
wherein an opposite, second end of the cam pin is configured to travel along the cam path.

6. The apparatus of claim 5, wherein at least one of the cam path or the second end of the cam pin is sized and shaped to prevent separation between the cam pin and the cam path.

7. The apparatus of claim 1, wherein the cutting tip comprises a lumen configured to receive at least of one of the tissue after being cut or the medical lead after being separated such that at least one of the tissue or the medical lead that enters the lumen has the lateral motion.

8. The apparatus of claim 7, wherein the lateral motion of the cutting tip is configured to cause a diameter of the tissue entering the lumen to be smaller than an inner diameter of the cutting tip.

9. A lead removal device, comprising:
a flexible elongate member configured to be positioned within a blood vessel of a patient, wherein the flexible elongate member comprises a proximal portion and a distal portion;
an outer band fixedly positioned at the distal portion of the flexible elongate member and comprising an inner surface having an inner diameter and a cam slot;
a cutting tip positioned at least partially within the outer band and configured to rotate with respect to the outer band, wherein the cutting tip comprises:
a blade configured to cut tissue associated with the blood vessel for removal of an electrical lead implanted in the tissue from a body of a patient;
an outer wall surface defining an outer diameter that is smaller than the inner diameter of the outer band, such that a gap exists between the outer wall surface and the inner surface of the outer band;
an inner wall surface defining a lumen with an inner diameter, wherein the lumen is configured to receive the electrical lead and the tissue cut by the blade; and
a guide pin fixedly attached to the outer wall surface and configured to slidably engage with the cam slot such that, proximate to the guide pin, the outer wall surface is in contact with the inner surface of the outer band,
wherein rotation of the cutting tip within the outer band causes the guide pin to slide within the cam slot, such that a center of rotation of the cutting tip is not aligned with a geometric center of the cutting tip, causing the cutting tip to move laterally as it rotates,
wherein a cutting edge of the blade is aligned with the inner wall surface,
such that a diameter of the tissue cut by the blade and entering the lumen is smaller than the inner diameter.

10. The lead removal device of claim 9, wherein a diameter of a region of tissue shredded by the cutting tip is equal to the inner diameter of the outer band.

11. The lead removal device of claim 9, wherein the cutting tip is configured such that at least a portion of tissue shredded by the cutting tip is received into the lumen.

12. The lead removal device of claim 9,
wherein the blade comprises a plurality of teeth and a plurality of serrations,
wherein a distal end of each tooth comprises a cutting edge.

13. The lead removal device of claim 12,
wherein each tooth of the plurality of teeth comprises a chamfered trapezoidal surface,
wherein each serration of the plurality of serrations comprises a chamfered curved surface.

14. The lead removal device of claim 9, further comprising:
a handle coupled to the proximal portion of the flexible elongate member; and,
a trigger coupled to the handle and configured such that actuation of the trigger causes the cutting tip to rotate.

15. The lead removal device of claim 14, wherein the trigger is coupled to a motor and a battery such that actuation of the trigger causes the motor to rotate the cutting tip.

16. The lead removal device of claim 15, wherein the trigger is coupled to the motor via a processor.

17. The lead removal device of claim 9, further comprising an outer jacket slidably coupled to the flexible elongate member such that when the outer jacket is advanced to a distal position, the blade is covered by the outer jacket, and when the outer jacket is retracted to a proximal location, the blade is not covered by the outer jacket.

18. The lead removal device of claim 9, wherein the cam slot is curved such that when the rotation of the cutting tip within the outer band causes the guide pin to slide within the cam slot, causing the cutting tip to move longitudinally.
